(19) United States
(12) Patent Application Publication
(10) Pub. No.: US 2023/0181443 A1
(43) Pub. Date: Jun. 15, 2023

(54) METHOD FOR TREATING HUMAN HAIR WITH AGENTS CONTAINING MIXTURES OF ORGANIC C1-C6 ALKOXY SILOXANES

(71) Applicant: Henkel AG & Co. KGaA, Duesseldorf (DE)

(72) Inventors: Phillip JAISER, Langenfeld (DE); Marc NOWOTTNY, Moenchengladbach (DE); Torsten LECHNER, Langenfeld (DE); Gabriele WESER, Essen (DE); Carsten MATHIASZYK, Essen (DE); Jing HODES, Hagen (DE); Andreas WALTER, Ratingen (DE); Juergen SCHOEPGENS, Schwalmtal (DE); Claudia KOLONKO, Remscheid (DE); Ulrike SCHUMACHER, Duesseldorf (DE); Caroline KRIENER, Duesseldorf (DE)

(73) Assignee: Henkel AG & Co. KGaA, Duesseldorf (DE)

(21) Appl. No.: 17/998,438

(22) PCT Filed: Mar. 29, 2021
(86) PCT No.: PCT/EP2021/058133

§ 371 Date: Nov. 10, 2022

(30) Foreign Application Priority Data
May 12, 2020 (DE) 10 2020 205 903.9

(51) Int. Cl. A61K 8/58; A61K 8/23; A61K 8/19; A61K 8/34; A61Q 5/06

(52) U.S. Cl. CPC A61K 8/585; A61K 8/23; A61K 8/19; A61K 8/34; A61Q 5/065; A61K 2800/882; A61K 2800/43

(57) ABSTRACT

The subject of the present application is a process for the treatment of keratinous material, in particular human hair, wherein a cosmetic agent is applied to the keratinous material and rinsed off again after a contact time, exemplified in that the cosmetic agent contains a mixture of organic C₁-C₆ alkoxy siloxanes which is obtained by mixing one or more organic C₁-C₆ alkoxysilanes with a solvent other than water and selectively hydrolyzing and precondensing the mixture by adding water and catalyst.

### CROSS REFERENCE TO RELATED APPLICATIONS

This application is a U.S. National-Stage entry under 35 U.S.C. § 371 based on International Application No. PCT/EP2021/058133, filed Mar. 29, 2021, which was published under PCT Article 21(2) and which claims priority to German Application No. 10 2020 205903.9, filed May 12, 2020, which are all hereby incorporated in their entirety by reference.

### TECHNICAL FIELD

The present application is in the field of cosmetics and concerns a process for treating human hair. In the process as contemplated herein, a cosmetic agent is applied to the hair and rinsed off again after a contact time. The cosmetic agent, which is an agent ready for use, is exemplified in that it contains a mixture of organic C₁-C₆ alkoxy siloxanes obtained by mixing one or more organic C₁-C₆ alkoxysilanes with a solvent other than water and selectively hydrolyzing and precondensing them by adding water and a catalyst.

A second subject matter is a process for treating, particularly preferably for coloring, keratinous material, in which first the ready-to-use cosmetic agent of the first subject of the present disclosure is prepared. For this purpose, the mixture of organic C₁-C₆ alkoxy siloxanes is mixed with another preparation, which is a water-containing cosmetic carrier preparation. This application mixture is then applied to the keratin material, left to act and rinsed out again. Optionally, a post-treatment agent can be applied afterwards.

A third object of the present disclosure is a multi-component packaging unit (kit-of-parts) for coloring keratinous material, which comprises the cosmetic preparations (A) and (B) separately packaged in two packaging units. Preparation (A) contains a mixture of organic C₁-C₆ alkoxy siloxanes, and preparation (B) is a water-containing cosmetic carrier formulation.

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dyeing with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing obtained with direct dyes have a shorter shelf life and quicker wash ability. Dyes with direct dyes usually remain on the hair for a period of between 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

EP 2168633 B1 deals with the task of producing long-lasting hair colorations using pigments. The document teaches that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these hydrolysis or condensation products are applied to keratinous material, a film or coating is formed on the keratinous material, which completely envelops the keratinous material and in this way strongly influences the properties of the keratinous material. Possible areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers, and surprisingly wash-resistant colorations result.

The great advantage of the alkoxy silane-based dyeing principle is that the high reactivity of this class of compounds enables very fast coating. This means that extremely good coloring results can be achieved after very short application periods of just a few minutes. In addition to these advantages, however, the high reactivity of alkoxy silanes also has some disadvantages. Thus, even minor changes in production and application conditions, such as changes in humidity and/or temperature, can lead to sharp fluctuations in product performance. Most importantly, the work leading to the present disclosure has shown that the alkoxy silanes are extremely sensitive to the conditions encountered in the manufacture of the keratin treatment agents.

Analytical studies have shown that complex hydrolysis and condensation reactions take place during the preparation of various silane or siloxane mixtures and blends, leading to oligomeric products of different molecular size depending on the reaction conditions selected. In this context, it has been found that the molecular weight of these silane oligomers can have a major influence on the subsequent product properties. If wrong conditions are selected during production, this can lead to the formation of silane condensates that are too large or too small, which negatively affects the subsequent product performance, especially the subsequent dyeing capacity on the keratin material.

It was the task of the present application to find an optimized process for the treatment of human hair. The mixtures of alkoxy-siloxanes used in this process were to be prepared in a targeted manner so that the optimum application properties could be achieved in a subsequent application. In particular, the agents prepared by this method should have improved dyeing performance, i.e., when used in a dyeing process, dyeing with higher color intensity and improved fastness properties, especially improved wash fastness and improved rub fastness, should be obtained.

Surprisingly, it has now been found that the above-mentioned task can be excellently solved if agents containing C₁-C₆ alkoxy-siloxane mixtures are applied to human hair, the C₁-C₆ alkoxy-siloxane mixtures being prepared in a special way using a catalyst.

### BRIEF SUMMARY

Methods for treating keratinous material and a kit-of-parts for the same are provided. In an exemplary embodiment, a method includes the steps of applying a cosmetic agent to the keratinous material, where the cosmetic agent includes an organic C₁-C₆ alkoxy siloxane formed by mixing an organic C₁-C₆ alkoxy silane with a solvent other than water. The C₁-C₆ alkoxy silane is selectively hydrolyzing and precondensed to form the organic C₁-C₆ alkoxy siloxane by adding water and a catalyst. The cosmetic agent is then rinsed off of the keratinous material after a contact time.

Another method for treating keratinous material is provided in another embodiment. The method includes providing a mixture of organic C₁-C₆ alkoxy siloxanes that is obtained by mixing an organic C₁-C₆ alkoxy silane with a solvent other than water, and then selectively hydrolyzing and precondensing the organic C₁-C₆ alkoxy silane to form the organic C₁-C₆ alkoxy siloxane by adding water and a catalyst. The mixture of the C₁-C₆ alkoxy siloxane is then blended with a water-containing cosmetic carrier to obtain a ready-to-use cosmetic agent. The ready-to-use cosmetic agent is applied to the keratinous material, which is exposed to the ready-to-use cosmetic agent. The ready-to-use cosmetic agent is rinsed from the keratinous material, and a post-treatment agent is then optionally applied to the keratinous material. The keratinous material is optionally exposed to the post-treatment agent, which is then the optionally rinsed off of the keratinous material.

A multicomponent packaging unit (a kit-of-parts) for treating keratinous material is provided in another embodiment. The kit-of-parts includes a first packaging unit containing a cosmetic preparation (A) and a second packaging unit containing a cosmetic preparation (B). The cosmetic preparation (A) includes an organic C₁-C₆ alkoxy siloxane that is obtained by mixing an organic C₁-C₆ alkoxy silane with a solvent other than water and selectively hydrolyzing and precondensing the organic C₁-C₆ alkoxy silane to form the organic C₁-C₆ alkoxy siloxane by adding water and a catalyst. The cosmetic preparation (B) includes a water-containing cosmetic carrier.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Here, the mixtures of organic C₁-C₆ alkoxy siloxanes are prepared by selective hydrolysis and condensation of organic C₁-C₆ alkoxysilanes. In the preparation of the mixture of organic C₁-C₆ alkoxy siloxanes, it has proved essential to the present disclosure to first mix the organic C₁-C₆ alkoxysilanes used for the preparation with a solvent other than water. Water is now added to this mixture of organic C₁-C₆ alkoxysilane and solvent to initiate targeted hydrolysis. Simultaneously with or shortly after hydrolysis, precondensation of the organic C₁-C₆ alkoxysilanes to the C₁-C₆ alkoxy-siloxane mixtures also follows. These C₁-C₆ alkoxy-siloxane mixtures represent oligomeric compounds that still possess reactive groups due to their partial hydrolysis or condensation and can react to form the final polymer, film or coating when applied to human hair. Good dyeing results with excellent fastness properties were obtained especially when the previously described hydrolysis and condensation were initiated and accelerated by using a catalyst.

A first object of the present disclosure is a process for the treatment of keratinous material, in particular human hair, wherein a cosmetic agent is applied to the keratinous material and rinsed off again after a contact time, exemplified in that the cosmetic agent contains a mixture of organic C₁-C₆ alkoxy siloxanes, which is obtained by mixing one or more organic C₁-C₆ alkoxysilanes with a solvent other than water and selectively hydrolyzing and precondensing the mixture by adding water and catalyst.

It was shown that the cosmetic agents containing the mixtures of organic C₁-C₆ alkoxy-siloxanes prepared in this special way, when used in a dyeing process, resulted in very intense and uniform colorations with very good hiding power, rub fastness and wash fastness.

### Keratinic Material

Keratinous material includes hair, skin, and nails (such as fingernails and/or toenails). Wool, furs and feathers also fall under the definition of keratinous material.

Preferably, keratinous material is understood to be human hair, human skin and human nails, especially fingernails and toenails. Keratinous material is understood to be human hair in particular.

### Agent for the Treatment of Keratinous Material

Agents for treating keratinous material are understood to mean, for example, techniques for coloring the keratinous material, techniques for reshaping or shaping keratinous material, in particular keratinous fibers, or also techniques for conditioning or caring for the keratinous material. The agents prepared via the process as contemplated herein show particularly good suitability for coloring keratinous material, in particular for coloring keratinous fibers, which are especially preferably human hair.

The term “coloring agent” is used in the context of the present disclosure to refer to a coloring of the keratin material, in particular of the hair, caused by the use of coloring compounds, such as thermochromic and photochromic dyes, pigments, mica, direct dyes and/or oxidation dyes. In this staining process, the aforementioned colorant compounds are deposited in a particularly homogeneous and smooth film on the surface of the keratin material or diffuse into the keratin fiber. The film is formed in situ by oligomerization or condensation of the organic silicon compound(s), with the colorant compound(s) interacting with or being incorporated into this film or coating.

As contemplated herein, the cosmetic agent, which is applied to the keratinous material, in particular human hair, and rinsed off again, constitutes an agent ready for use. This ready-to-use agent can, for example, be filled into a container and applied to the keratin material in this form without further dilution, mixing or other process steps. For reasons of storage stability, however, it has been found to be particularly preferable if the cosmetic agent ready for use is prepared by the hairdresser or user only shortly before application. For this preparation of the ready-to-use agent, for example, the mixture of organic C₁-C₆ alkoxy siloxanes, which is provided in the form of a separately prepared concentrate, can be mixed with a water-containing cosmetic carrier formulation.

Furthermore, it is also possible to prepare the ready-to-use cosmetic agent by mixing three different preparations, the first preparation containing the mixture of organic C₁-C₆ alkoxy siloxanes, the second preparation representing the water-containing carrier, and the third preparation being able to contain further active ingredients or ingredients such as, for example, colorants, thickeners or acids and/or bases for adjusting the desired pH value.

Organic C₁-C₆ Alkoxy Siloxanes

By siloxanes, the skilled person understands chemical compounds with the general formula R₃Si—[O—SiR₂]n-O—SiR₃, where R, can be hydrogen atoms, alkyl groups or also substituted alkyl groups. In siloxanes, the silicon atoms are each linked to their neighboring silicon atom by an oxygen atom: Si—O—Si. Siloxanes with R═CH₃ are called polydimethylsiloxanes. The index n indicates the degree of oligomerization or polymerization of the siloxane. Usually the index n is a number from 0 to about 1,000,000, or from 0 to about 100,000, or from 0 to about 10,000, or from 0 to about 1,000. When n is 0, the siloxane is in the form of a dimer.

The organic C₁-C₆ alkoxy siloxanes of the present disclosure are exemplified in that at least one radical R is a C₁-C₆ alkoxy group. The C₁-C₆ alkoxy group attached to the silicon atom represents a reactive leaving group which, when applied to the keratin material, enables further condensation or oligomerization or polymerization.

The organic C₁-C₆ alkoxy siloxanes are prepared by mixing one or more organic C₁-C₆ alkoxysilanes with a solvent other than water and selectively hydrolyzing and precondensing them by adding water and catalyst.

Organic C₁-C₆ Alkoxy Silanes

The organic C₁-C₆ alkoxy silane(s) are organic, non-polymeric silicon compounds, preferably selected from the group of silanes containing one, two or three silicon atoms.

Organic silicon compounds, alternatively called organosilicone compounds, are compounds which either have a direct silicon-carbon bond (Si—C) or in which the carbon is bonded to the silicon atom via an oxygen, nitrogen or sulfur atom. The organic silicon compounds of the present disclosure are preferably compounds containing one to three silicon atoms. Organic silicon compounds preferably contain one or two silicon atoms.

According to IUPAC rules, the term silane chemical compounds are based on a silicon skeleton and hydrogen. In organic silanes, the hydrogen atoms are completely or partially replaced by organic groups such as (substituted) alkyl groups and/or alkoxy groups.

An exemplary feature of the C₁-C₆ alkoxy silanes of the present disclosure is that at least one C₁-C₆ alkoxy group is directly bonded to a silicon atom. The C₁-C₆ alkoxy silanes as contemplated herein thus comprise at least one structural unit R′R″R′″Si—O—(C₁-C₆-Alkyl) where the radicals R′, R″ and R′″ represent the three remaining bond valencies of the silicon atom.

The C₁-C₆ alkoxy group or groups bonded to the silicon atom are very reactive and are hydrolyzed at high rates in the presence of water, the reaction rate depending, among other things, on the number of hydrolysable groups per molecule. If the hydrolysable C₁-C₆ alkoxy group is an ethoxy group, the organic silicon compound preferably contains a structural unit R′R″R′″Si—O—CH2-CH3. The radicals R′, R″ and R′″ again represent the three remaining free valences of the silicon atom.

In a very particularly preferred embodiment, the process as contemplated herein uses a cosmetic agent comprising a mixture of C₁-C₆ alkoxy siloxanes, in the preparation of which one or more organic C₁-C₆ alkoxysilanes of the formula (I) and/or (II) and/or (IV) are used.

In a very particularly preferred embodiment, a process as contemplated herein is exemplified in that the cosmetic agent comprises a mixture of organic C₁-C₆ alkoxy-siloxanes obtained by mixing one or more organic C₁-C₆ alkoxysilanes of the formula (I) and/or (II) and/or (IV) with the solvent

R₁R₂N-L-Si(OR₃)ₐ(R₄)_{b} (I)

where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a, stands for an integer from 1 to 3, and
b stands for the integer 3-a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),

-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h radicals are different from 0,

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

Particularly preferred is the use of organic C₁-C₆ alkoxy silanes of the formula (I) and/or of the formula (II),

R₁R₂N-L-Si(OR₃)ₐ(R₄)_{b} (I)

where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a, stands for an integer from 1 to 3, and
b stands for the integer 3-a, and

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),

-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of the radicals e, f, g and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the amino methyl group, the 2-aminoethyl group, the 3-aminopropyl group. The 2-aminoethyl group is particularly preferred. Examples of a linear bivalent C₁-C₂₀ alkylene group include the methylene group (—CH₂—), the ethylene group (—CH₂—CH₂—), the propylene group (—CH₂—CH₂—CH₂—), and the butylene group (—CH₂—CH₂—CH₂—CH₂—). The propylene group (—CH₂—CH₂—CH₂—) is particularly preferred. From a chain length of 3 C atoms, bivalent alkylene groups can also be branched. Examples of branched divalent, bivalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. Most preferably, the radicals R₁ and R₂ both represent a hydrogen atom.

In the middle part of the organic silicon compound is the structural unit or the linker -L- which stands for a linear or branched, divalent C₁-C₂₀ alkylene group. The divalent C₁-C₂₀ alkylene group may alternatively be referred to as a divalent or divalent C₁-C₂₀ alkylene group, by which is meant that each -L- grouping may form two bonds.

Preferably -L- stands for a linear, bivalent C₁-C₂₀ alkylene group. Further preferably -L- stands for a linear bivalent C₁-C₆ alkylene group. Particularly preferred -L-stands for a methylene group (—CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

one end of each carries the silicon-containing group —Si(OR₃)ₐ(R₄)_{b}.

In the terminal structural unit —Si(OR₃)ₐ(R₄)_{b} radicals R₃ and R₄ independently represent a C₁-C₆ alkyl group, and particularly preferably R₃ and R₄ independently represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Keratin treatment agents with particularly good properties could be prepared if in step (1) at least one organic C₁-C₆ alkoxy silane of formula (I) was mixed with water or reacted, in which the radicals R₃, R₄ independently of one another represent a methyl group or an ethyl group.

Furthermore, dyeing with the best wash fastness could be obtained when at least one organic C₁-C₆ alkoxy silane of formula (I) was reacted with water in step (1), in which the radical a represents the number 3. In this case the radical b stands for the number 0.

In another preferred embodiment, a process as contemplated herein is exemplified in that in step (1) one or more organic C₁-C₆ alkoxy silanes of formula (I) are mixed with water, where
R₃, R₄ independently of one another represent a methyl group or an ethyl group and
a stands for the number 3 and
b stands for the number 0.

In a further preferred embodiment, a process as contemplated herein is exemplified in that the cosmetic agent comprises a mixture of organic C₁-C₆ alkoxy siloxanes obtained by mixing one or more organic C₁-C₆ alkoxysilanes of the formula (I) with a solvent other than water and selectively hydrolyzing and precondensing them by adding water and catalyst,

R₁R₂N-L-Si(OR₃)ₐ(R₄)_{b} (I),

where
R₁, R₂ both represent a hydrogen atom, and
L represents a linear, bivalent C₁-C₆-alkylene group, preferably a propylene group (—CH₂—CH₂—CH₂—) or an ethylene group (—CH₂—CH₂—),
R₃ represents an ethyl group or a methyl group,
R₄ represents a methyl group or an ethyl group,
a stands for the number 3 and
b stands for the number 0.

Organic silicon compounds of the formula (I) which are particularly suitable for solving the problem as contemplated herein are

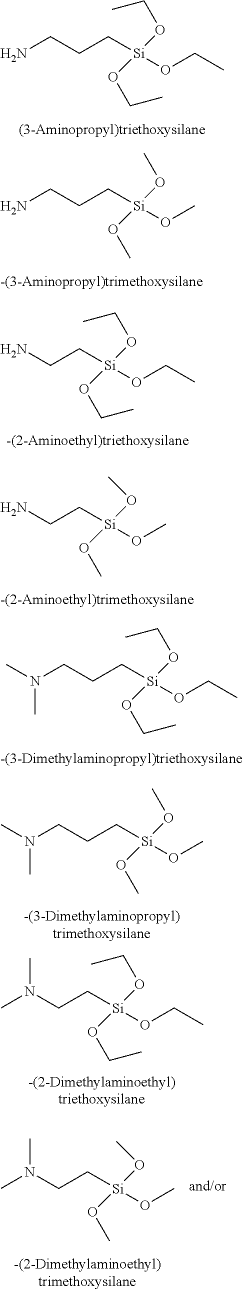

In another preferred embodiment, a process as contemplated herein is exemplified in that the cosmetic agent comprises a mixture of organic C₁-C₆ alkoxy siloxanes obtained by adding one or more organic C₁-C₆ alkoxysilanes selected from the group of
(3-Aminopropyl)triethoxysilane
(3-Aminopropyl)trimethoxysilane
1-(3-Aminopropyl)silantriol
(2-Aminoethyl)triethoxysilane
(2-Aminoethyl)trimethoxysilane
1-(2-Aminoethyl)silantriol
(3-Dimethylaminopropyl)triethoxysilane
(3-Dimethylaminopropyl)trimethoxysilane
1-(3-Dimethylaminopropyl)silantriol
(2-dimethylaminoethyl)triethoxysilane.
(2-Dimethylaminoethyl)trimethoxysilane and/or
1-(2-Dimethylaminoethyl)silantriol
   mixed with a solvent different from water and selectively hydrolyzed and precondensed by adding water and catalyst.

The aforementioned organic silicon compound of formula (I) is commercially available. (3-aminopropyl)trimethoxysilane, for example, can be purchased from Sigma-Aldrich®. Also (3-aminopropyl)triethoxysilane is commercially available from Sigma-Aldrich®.

In a further preferred embodiment, a process as contemplated herein is exemplified in that the cosmetic agent comprises a mixture of organic C₁-C₆ alkoxy siloxanes obtained by mixing one or more organic C₁-C₆ alkoxysilanes of the formula (II) with a solvent other than water and selectively hydrolyzing and precondensing them by adding water and catalyst,

(R₅O)_{c}(R₆)_{d}Si-(A)ₑ-[NR₇-(A′)]O—[O-(A″)]_{g}-[NR₈-(A′″)]ₕ-Si(R₆′)_{d′}(OR₅′)_{c′} (II).

The organosilicon compounds of formula (II) as contemplated herein each carry the silicon-containing groups (R₅O)_{c}(R₆)_{d}Si— and —Si(R₆′)_{d′}(OR₅′)_{c′} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) as contemplated herein contains at least one grouping from the group of -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}′, the radicals R5, R5′, R5″ independently represent a C₁-C₆ alkyl group. The radicals R6, R6′ and R6″ independently represent a C₁-C₆ alkyl group.

Here c stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

Dyeing with the best wash fastness values could be obtained if the radicals c and c′ both stand for the number 3. In this case d and d′ both stand for the number 0.

In a further preferred embodiment, a process as contemplated herein is exemplified in that the cosmetic agent comprises a mixture of organic C₁-C₆ alkoxy siloxanes obtained by mixing one or more organic C₁-C₆ alkoxysilanes of the formula (II) with a solvent other than water and selectively hydrolyzing and precondensing them by adding water and catalyst,

(R₅O)_{c}(R₆)_{d}Si-(A)ₑ-[NR₇-(A′)]O—[O-(A″)]_{g}-[NR₈-(A′″)]ₕ-Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]O—[O-(A″)]_{g}-[NR₈-(A′″)]ₕ-Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and —[NR₇-(A′)]_{f}- and —[O-(A″)]_{g}- and —[NR₈-(A′″)]ₕ- are located in the middle part of the organic silicon compound of formula (II).

In this context, the presence of certain groupings has proven to be particularly advantageous in terms of achieving washfast dyeing results. Particularly good results could be obtained if at least two of the radicals e, f, g and h stand for the number 1. Especially preferred e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A″″ independently represent a linear or divalent, bivalent C₁-C₂₀ alkylene group. Preferably the radicals A, A′, A″, A′″ and A″″ independently of one another represent a linear, bivalent C₁-C₂₀ alkylene group. Further preferably the radicals A, A′, A″, A′″ and A″″ independently represent a linear bivalent C₁-C₆ alkylene group.

The divalent C₁-C₂₀ alkylene group may alternatively be referred to as a divalent or divalent C₁-C₂₀ alkylene group, by which is meant that each grouping A, A′, A″, A′″ and A″″ may form two bonds.

In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). Very preferably, the radicals A, A′, A″, A′″ and A″″ represent a propylene group (—CH₂—CH₂—CH₂—).

If the radical f represents the number 1, then the organic silicon compound of formula (II) as contemplated herein contains a structural grouping —[NR₇-(A′)]-. If the radical h represents the number 1, then the organic silicon compound of formula (II) as contemplated herein contains a structural grouping —[NR₈-(A′″)]-.

Wherein radicals R₇ and R₈ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III).

Very preferably the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound as contemplated herein contains the grouping [NR₇-(A′)] but not the grouping —[NR₈-(A′″)]. If the radical R7 now stands for a grouping of the formula (III), the pretreatment agent (a) contains an organic silicon compound with 3 reactive silane groups.

In a further preferred embodiment, a process as contemplated herein is exemplified in that the cosmetic agent comprises a mixture of organic C₁-C₆ alkoxy siloxanes obtained by mixing one or more organic C₁-C₆ alkoxysilanes of the formula (II) with a solvent other than water and selectively hydrolyzing and precondensing them by adding water and catalyst,

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, a process as contemplated herein is exemplified in that the cosmetic agent comprises a mixture of organic C₁-C₆ alkoxy-siloxanes which is obtained by mixing one or more organic C₁-C₆ alkoxysilanes of the formula (II) with a solvent other than water and selectively hydrolyzing and precondensing them by adding water and catalyst, wherein
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂), and
R7 represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem as contemplated herein are

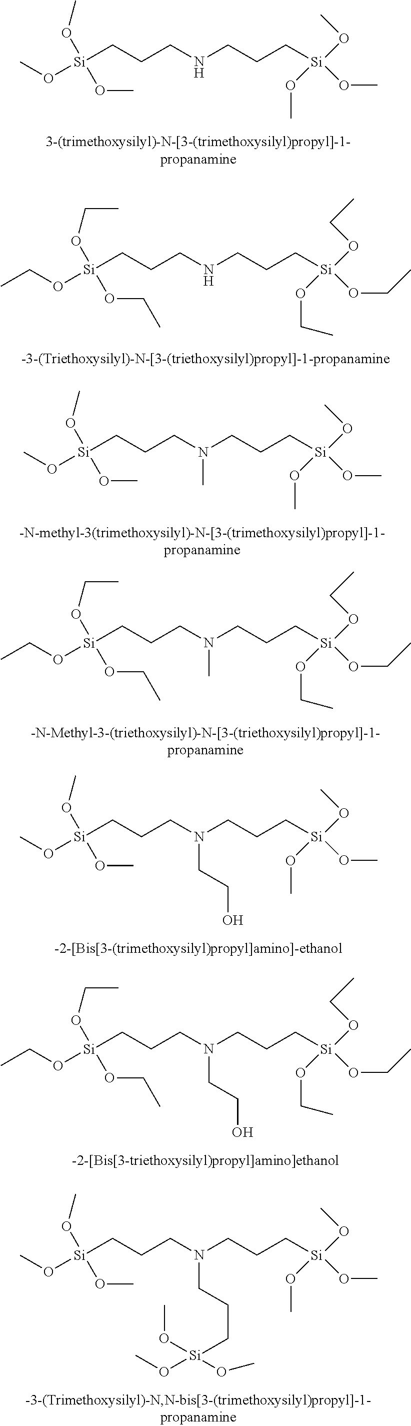

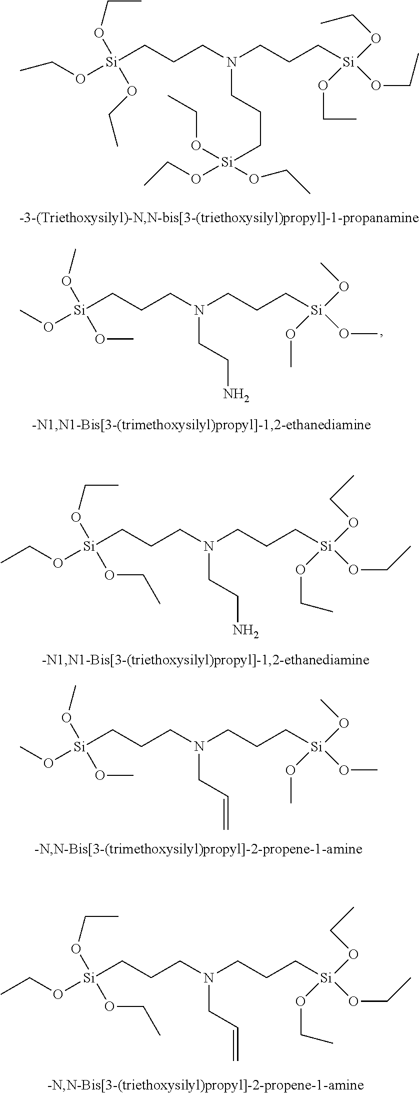

The aforementioned organic silicon compounds of formula (II) are commercially available.

Bis(trimethoxysilylpropyl)amines with the CAS number 82985-35-1 can be purchased from Sigma-Aldrich®.
Bis[3-(triethoxysilyl)propyl]amines with the CAS number 13497-18-2 can be purchased from Sigma-Aldrich®, for example.
N-methyl-3-(trimethoxysilyl)-N-[3-(trimethoxysilyl)propyl]-1-propanamine is alternatively referred to as Bis(3-trimethoxysilylpropyl)-N-methylamine and can be purchased commercially from Sigma-Aldrich® or Fluorochem®. 3-(triethoxysilyl)-N,N-bis[3-(triethoxysilyl)propyl]-1-propanamine with the CAS number 18784-74-2 can be purchased for example from Fluorochem® or Sigma-Aldrich®.

In another preferred embodiment, a process as contemplated herein is exemplified in that the cosmetic agent comprises a mixture of organic C₁-C₆ alkoxy siloxanes obtained by adding one or more organic C₁-C₆ alkoxysilanes selected from the group of
3-(Trimethoxysilyl)-N-[3-(Trimethoxysilyl)propyl]-1-propanamine
3-(Triethoxysilyl)-N-[3-(triethoxysilyl)propyl]-1-propanamine
N-Methyl-3-(trimethoxysilyl)-N-[3-(trimethoxysilyl)propyl]-1-propanamine
N-Methyl-3-(triethoxysilyl)-N-[3-(triethoxysilyl) propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilylpropyl]-1,2-ethanediamine,
N,N-Bis[3-(trimethoxysilyl)propyl]-2-Propen-1-amine and/or
N,N-Bis[3-(triethoxysilyl)propyl]-2-propen-1-amine,
   mixed with a solvent different from water and selectively hydrolyzed and precondensed by adding water and catalyst.

In further dyeing trials, it has also proved to be particularly advantageous, when the cosmetic agent comprises a mixture of organic C₁-C₆ alkoxy siloxanes obtained by mixing one or more organic C₁-C₆ alkoxysilanes of formula (IV) with a solvent other than water and selectively hydrolyzing and precondensing them by the addition of water and catalyst

(R₉)ₘSi(OR₁₀)ₖ (IV).

The compounds of formula (IV) are organic silicon compounds selected from silanes having one, two or three silicon atoms, wherein the organic silicon compound comprises one or more hydrolysable groups per molecule.

The organic silicon compound(s) of formula (IV) may be designated as silanes of the C₁-C₁₂ alkyl-C₁-C₆ alkoxy silane type (in the case of k=1 to 3) or as silanes of the tetra-C₁-C₆ alkoxy silane type (in the case of k=4),

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the integer 4-k.

In a further preferred embodiment, a process as contemplated herein is exemplified in that the cosmetic agent comprises a mixture of organic C₁-C₆ alkoxy siloxanes obtained by mixing one or more organic C₁-C₆ alkoxysilanes of the formula (IV) with a solvent other than water and selectively hydrolyzing and precondensing them by adding water and catalyst,

(R₉)ₘSi(OR₁₀)ₖ (IV),

where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

In the organic C₁-C₆ alkoxy silanes of formula (IV), the radical R₉ is a C₁-C₁₂ alkyl group or a C₂-C₁₂ alkenyl group. This C₁-C₁₂ alkyl group is saturated and can be linear or branched. The C₂-C₁₂ alkenyl group may comprise one or more double bonds and may be linear or branched. Preferably R₉ stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a C₁-C₆ alkyl group. In particular, R10 stands for a methyl group or an ethyl group.

Furthermore, k represents an integer from 1 to 4, and m represents the integer 4-k.

If k stands for the number 4, m is equal to 0. In this case, the silanes of formula (IV) are tetra-C₁-C₆ alkoxy silanes. Suitable silanes of this type are, for example, tetreethoxysilane or tetramethoxysilane.

If k stands for the number 3, m is equal to 1. In this case, the silanes of formula (IV) are C₁-C₁₂ alkyl tri-C₁-C₆ alkoxy silanes.

If k stands for the number 2, m is equal to 2. In this case, the silanes of formula (IV) are di-C₁-C₁₂-alkyl-di-C₁-C₆-alkoxy silanes.

If k stands for the number 1, m is equal to 3. In this case, the silanes of formula (IV) are tri-C₁-C₁₂-alkyl-C₁-C₆-alkoxy silanes.

Dyeing with the best wash fastness could be obtained if at least one organic silicon compound of formula (IV), in which the radical k represents the number 3, was used in the preparation of the preparation as contemplated herein. In this case the radical m stands for the number 1.

Furthermore, particularly good results were obtained when at least one organic silicon compound of the formula (IV) was used in the preparation of the preparation as contemplated herein, in which the radical R9 is a C₁-C₈ alkyl group and the radical R10 is a methyl group or an ethyl group.

In another very particularly preferred embodiment, a process as contemplated herein is exemplified in that the cosmetic agent comprises a mixture of organic C₁-C₆ alkoxy siloxanes obtained by mixing one or more organic C₁-C₆ alkoxysilanes of the formula (IV) with a solvent other than water and selectively hydrolyzing and precondensing them by adding water and catalyst,

(R₉)ₘSi(OR₁₀)ₖ (IV),

where
R₉ represents a C₁-C₈ alkyl group,
R₁₀ represents a methyl group or an ethyl group,
k stands for the number 3, and
m stands for the number 1.

Organic silicon compounds of the formula (IV) which are particularly suitable for solving the problem as contemplated herein are

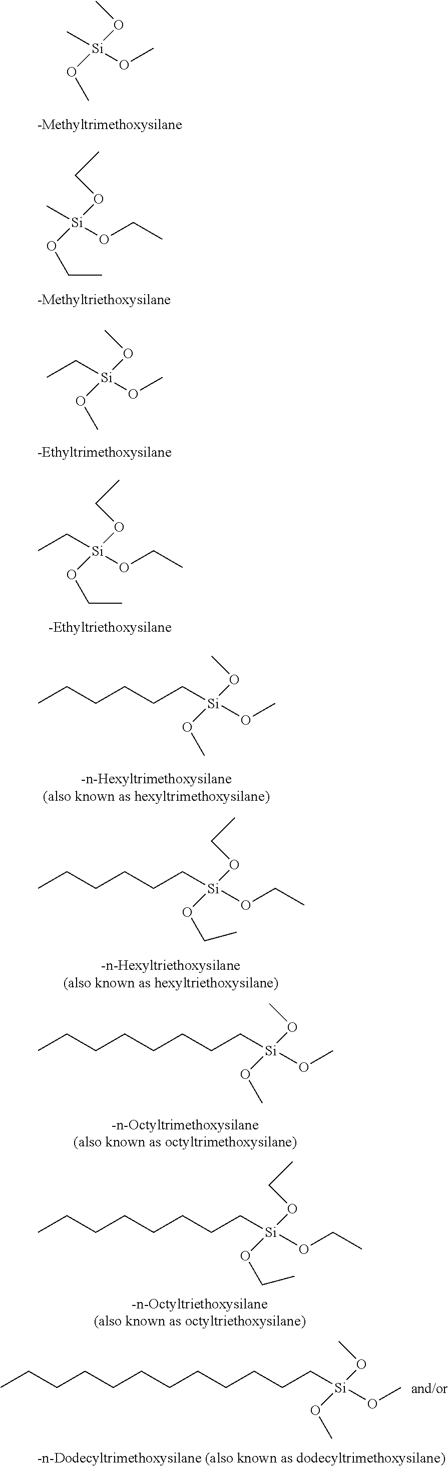

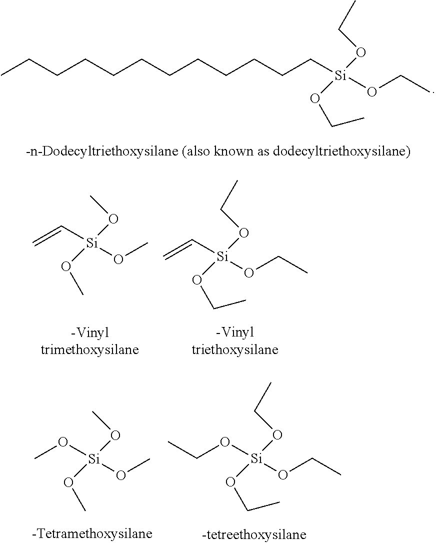

In another preferred embodiment, a process as contemplated herein is exemplified in that the cosmetic agent comprises a mixture of organic C₁-C₆ alkoxy siloxanes obtained by adding one or more organic C₁-C₆ alkoxysilanes selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane and/or
Dodecyltriethoxysilane,
Vinyl trimethoxysilane
Vinyl triethoxysilane
Tetramethoxysilane
Tetraethoxysilane
   mixed with a solvent different from water and selectively hydrolyzed and precondensed by adding water and catalyst.

In summary, in another preferred embodiment, a process as contemplated herein is exemplified in that the cosmetic agent comprises a mixture of organic C₁-C₆ alkoxy siloxanes obtained by mixing with the solvent one or more organic C₁-C₆ alkoxysilanes selected from the group of
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Vinyl trimethoxysilane
Vinyl triethoxysilane
Tetramethoxysilane
Tetraethoxysilane
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane, and
(2-Dimethylaminoethyl)trimethoxysilane.

In the preparation of the mixture of C₁-C₆ alkoxy-siloxanes, only one organic C₁-C₆ alkoxysilane from the group of compounds of the formula (I), only one organic C₁-C₆ alkoxysilane from the group of compounds of the formula (II), or also only one organic C₁-C₆ alkoxysilane from the group of compounds of the formula (IV) can be used.

However, cosmetic agents with particularly advantageous properties were obtained when mixtures of different C₁-C₆ alkoxy-silanes were also used in the preparation of the mixture of C₁-C₆ alkoxy-siloxanes. Particularly good results were obtained when both at least one C₁-C₆ organic alkoxysilane of formula (I) and at least one C₁-C₆ organic alkoxysilane of formula (IV) were used. When applied to the keratin material, corresponding agents led to the formation of particularly flexible and resistant coatings or films.

In this context, it was particularly advantageous to use the organic C₁-C₆ alkoxysilanes of the formula (I) and the organic C₁-C₆ alkoxysilane of the formula (IV) in certain ratios to one another.

In another particularly preferred embodiment, a process as contemplated herein is exemplified in that the cosmetic agent comprises a mixture of organic C₁-C₆ alkoxy-siloxanes obtained by mixing one or more organic C₁-C₆ alkoxysilanes of formula (I) and one or more organic C₁-C₆ alkoxysilanes of formula (IV) in a weight ratio of (I)/(IV) of about 1:1 to about 1:10, preferably from about 1:1 to about 1:8, more preferably from about 1:1 to about 1:6, still more preferably from about 1:1 to about 1:4 and most preferably from about 1:2 to about 1:4 to each other.

At a weight ratio of the organic C₁-C₆ alkoxysilanes of the formula (I) and the organic C₁-C₆ alkoxysilanes of the formula (IV), i.e. at a weight ratio (I)/(IV), of 1:1, for example, 1 part by weight of (3-aminopropyl)triethoxysilane and 1 part by weight of methyltriethoxysilane can be used. Furthermore, 1 part by weight of (3-aminopropyl)triethoxysilane and 1 part by weight of methyltrimethoxysilane can also be used.

At a weight ratio of the organic C₁-C₆ alkoxysilanes of the formula (I) and the organic C₁-C₆ alkoxysilanes of the formula (IV), i.e. at a weight ratio (I)/(IV), of 1:10, for example, 1 part by weight of (3-aminopropyl)triethoxysilane and 10 parts by weight of methyltriethoxysilane can be used. Furthermore, 1 part by weight of (3-aminopropyl)triethoxysilane and 10 parts by weight of methyltrimethoxysilane can also be used.

The stated weight ratios are understood to be the total amount of all organic C₁-C₆ alkoxysilanes of formula (I) used in the preparation of the mixture of organic C₁-C₆ alkoxysilanes, which is set in relation to the total amount of all organic C₁-C₆ alkoxysilanes of formula (IV).

Very preferably, the weight ratio of (I)/(IV) is from about 1:1 to about 1:8, more preferably from about 1:1 to about 1:6, still more preferably from about 1:1 to about 1:4, and most preferably from about 1:2 to about 1:4.

In other words, it has been found to be particularly preferred if the organic C₁-C₆ alkoxysilanes of the formula (IV) are used in a two- to fourfold excess by weight compared with the organic C₁-C₆ alkoxysilanes of the formula (I).

Furthermore, good results were also obtained when both at least one C₁-C₆ organic alkoxysilane of formula (I) and at least one C₁-C₆ organic alkoxysilane of formula (II) were used. In this context, it was advantageous to use the organic C₁-C₆ alkoxysilanes of the formula (I) and the organic C₁-C₆ alkoxysilane of the formula (II) in certain proportions to one another.

In a further embodiment, a process as contemplated herein is exemplified in that the cosmetic agent comprises a mixture of organic C₁-C₆ alkoxy-siloxanes obtained by mixing one or more organic C₁-C₆ alkoxysilanes of formula (I) and one or more organic C₁-C₆ alkoxysilanes of formula (II) in a weight ratio of (I)/(II) of about 1:1 to about 1:10, preferably from about 1:1 to about 1:8, more preferably from about 1:1 to about 1:6, still more preferably from about 1:1 to about 1:4 and most preferably from about 1:2 to about 1:4 to each other.

### Reaction Vessels

The preparation of the mixture of organic C₁-C₆ alkoxysiloxanes is preferably carried out in a reactor or reaction vessel suitable for this purpose. A reaction vessel that is very suitable for smaller preparations is, for example, a glass flask commonly used for chemical reactions with a capacity of about 1 liter, about 3 liters or about 5 liters, such as a 3-liter single-neck or multi-neck flask with ground joints.

A reactor is a confined space (container, vessel) that has been specially designed and manufactured to allow certain reactions to take place and be controlled under defined conditions.

For larger approaches, it has proven advantageous to carry out the reaction in reactors made of metal. Typical reactors may include, for example, about a 10-liter, 20-liter, or 50-liter capacity. Larger reactors for the production area can also include fill volumes of about 100-liters, about 500-liters or about 1000-liters.

Double-wall reactors have two reactor shells or reactor walls, with a tempering fluid circulating in the area between the two walls. This enables particularly good adjustment of the temperature to the required values.

The use of reactors, in particular double-walled reactors with an enlarged heat exchange surface, has also proven to be particularly suitable, whereby the heat exchange can take place either through internal installations or through the use of an external heat exchanger.

Corresponding reactors are, for example, laboratory reactors from the company IKA. In this context, the models “LR-2.ST” or the model “magic plant” can be mentioned.

Other reactors that can be used are reactors with thin-film evaporators, since this allows very good heat dissipation and thus particularly precise temperature control. Thin film evaporators are alternatively referred to as thin film evaporators. Thin film evaporators can be purchased commercially from Asahi Glassplant® Inc. for example.

### Solvent

In the preparation of the mixture of organic C₁-C₆ alkoxy-siloxanes, the organic C₁-C₆ alkoxysilane or organic C₁-C₆ alkoxysilanes, in particular the aforementioned preferred and especially preferred representatives, are mixed with a solvent other than water in a first step.

This step has proven to be essential in order to create a uniform film on the keratin material during the subsequent application of the cosmetic agents to the keratin material. As a result of the higher uniformity of the film—if the process as contemplated herein is, for example, a dyeing process—particularly uniform dyeing with a high leveling capacity can be produced.

Mixing can be accomplished, for example, by first placing the solvent other than water in a suitable reactor or reaction vessel and then adding the organic C₁-C₆ alkoxysilane(s). The addition can be done by dripping or pouring. Furthermore, it is also possible and as contemplated herein if at least one organic C₁-C₆ alkoxysilane is first introduced into the reaction vessel and then the solvent is added or added dropwise.

A sequential approach is also possible, i.e. first the addition of solvent and a first organic C₁-C₆ alkoxysilane, then again the addition of a solvent and then again the addition of another organic C₁-C₆ alkoxysilane.

The solvent is preferably added with stirring.

It may be preferred to select a solvent that has a boiling point at normal pressure (1013 hPa) of from about 20 to about 90° C., preferably from about 30 to about 85° C., and most preferably from about 40 to about 80° C.

Suitable solvents include:
Dichloromethane with a boiling point of 40° C. (1013 mbar)
Methanol with a boiling point of 65° C. (1013 mbar)
Tetrahydrofuran with a boiling point of 65.8° C. (1013 mbar)
Ethanol with a boiling point of 78° C. (1013 mbar)
Isopropanol with a boiling point of 82° C. (1013 mbar)
Acetonitrile with a boiling point of 82° C. (1013 mbar)

Furthermore, very particularly preferred solvents can be selected from the group of monohydric or polyhydric C₁-C₁₂ alcohols. Monohydric or polyhydric C₁-C₁₂ alcohols are compounds containing one to twelve carbon atoms and bearing one or more hydroxyl groups. Other functional groups different from the hydroxy groups are not present in the C₁-C₁₂ alcohols as contemplated herein. The C₁-C₁₂ alcohols can be aliphatic or aromatic.

Suitable C₁-C₁₂ alcohols may include methanol, ethanol, n-propanol, isopropanol, n-pentanol, n-hexanol, benzyl alcohol, 2-phenylethanol, 1,2-propanediol, 1,3-propanediol and glycerol. Particularly suitable C₁-C₁₂ alcohols are methanol, ethanol and isopropanol.

In another particularly preferred embodiment, a process as contemplated herein is exemplified in that the solvent other than water is selected from the group of mono- or polyhydric C₁-C₁₂ alcohols, preferably from the group of methanol, ethanol and isopropanol.

After the mixture of organic C₁-C₆ alkoxy siloxanes has been prepared, the solvent can be removed again. Removal can be accomplished by distillation off under reduced pressure, for example using a rotary evaporator. However, further work has shown that it is particularly advantageous to leave the solvent(s) in the mixture of C₁-C₆ organic alkoxy siloxanes.

If, for example, the mixture of C₁-C₆ alkoxy siloxanes, which still contains appropriate amounts of solvent, is used in a process for coloring keratin material, the cosmetic agent to be used will continue to contain at least one coloring compound, in particular at least one pigment. It has now been found that the solvents still contained in the mixture of C₁-C₆ alkoxy siloxanes allow particularly good mixing of pigments with the siloxanes, so that the pigments can be integrated particularly well into the coating formed on the keratin material. In this way, the film or coating is colored particularly evenly, and the color absorption, i.e. the integration of the pigments into the film, is also particularly good. This observation was made particularly when a C₁-C₁₂ alcohol, especially preferably ethanol, isopropanol or methanol, was used as the solvent.

To ensure uniform formation of the film on the keratin material, it has further proven to be particularly advantageous to use the solvent or solvents in certain quantity ranges in the preparation of the mixture of C₁-C₆ alkoxy siloxanes.

Particularly good results were obtained when the cosmetic agent contained a mixture of organic C₁-C₆ alkoxy siloxanes obtained by using one or more organic C₁-C₆ alkoxysilanes and the solvent in a weight ratio of from about 5:1 to about 1:5, preferably from about 5:1 to about 1:1, more preferably from about 5:1 to about 2:1 and most preferably from about 4:1 to about 2:1 to each other.

In another particularly preferred embodiment, a process as contemplated herein is exemplified in that the cosmetic agent comprises a mixture of organic C₁-C₆ alkoxy siloxanes obtained by using one or more organic C₁-C₆ alkoxysilanes and a solvent other than water in a weight ratio of from about 5:1 to about 1:5, preferably from about 5:1 to about 1:1, more preferably from about 5:1 to about 2:1 and most preferably from about 4:1 to about 2:1 to one another.

Under the weight ratio of C₁-C₆ organic alkoxysilanes to the solvent is to be understood as the total amount of C₁-C₆ alkoxysilanes used in the production, which is set in relation to the total amount of solvent other than water used.

For example, at a weight ratio of the organic C₁-C₆ alkoxysilanes to the solvent of 4:1 to 2:1, it is understood that the organic C₁-C₆ alkoxysilanes are used in a fourfold to twofold weight excess compared to the solvent.

The amount of solvents means all the solvents that are mixed with the organic C₁-C₆ alkoxysilanes during the preparation of the mixture of organic C₁-C₆ alkoxy-siloxanes.

### Targeted Hydrolysis by Addition of Water

Water is now added to the mixture of one or more organic C₁-C₆ alkoxysilanes, preferably those of formula (I), (II) and/or (IV), and the solvent, which is different from water, in order to initiate a specific hydrolysis and, as a result, a precondensation.

Water can be added, for example, by dropping or pouring the water into the mixture of the C₁-C₆ organic alkoxysilanes and the solvent.

In this case, the dripping or the addition of the water can be done at room temperature. However, it is particularly advantageous for the application properties of the subsequent cosmetic agent if the mixture of organic C₁-C₆ alkoxysilanes and solvent is heated to a temperature of from about 30 to about 80° C., preferably from about 40 to about 75° C., further preferably from about 45 to about 70° C. and most preferably from about 50 to about 65° C., before the water and catalyst are added.

In another particularly preferred embodiment, a process as contemplated herein is exemplified in that the mixture of organic C₁-C₆ alkoxysilanes and solvent is heated to a temperature of from about 30 to about 80° C., preferably from about 40 to about 75° C., more preferably from about 45 to about 70° C., and most preferably from about 50 to about 65° C., before water and catalyst are added.

Adjustment of the preferred and particularly preferred temperature ranges can be accomplished by tempering the reaction vessel or reactor. For example, the reaction vessel or reactor may be surrounded from the outside by a temperature control bath, which may be a water bath or silicone oil bath, for example.

If the reaction is carried out in a double-walled reactor, a temperature-controlled liquid can also be passed through the space formed by the two walls surrounding the reaction chamber.

Since the hydrolysis reaction is exothermic, it has been found to be particularly advantageous to stir or mix the reaction mixture for improved heat dissipation. It is therefore particularly preferred that the water be added while stirring. The reaction, which is now initiated by the addition of water and catalyst, continues to proceed exothermically, so that the reaction mixture remains at the preferred temperature ranges indicated above or may even heat up further without any further addition of energy. It is preferred if the additional heating due to the exothermic nature of the reaction remains within a range of about 5 to about 20° C. If the reaction mixture heats up beyond this range, it is advantageous to cool the mixture.

The water can be added continuously, in partial quantities or directly as a total quantity. To ensure adequate temperature control, the reaction mixture is adjusted for the amount and rate of water added. Depending on the amount of silanes used, the addition and reaction can take place over a period of about 2 minutes to about 72 hours.

The addition of the water initiates a selective hydrolysis of the organic C₁-C₆ alkoxysilanes. For the purposes of the present disclosure, targeted hydrolysis means hydrolyzing some, but not all, of the C₁-C₆ alkoxy groups present in the C₁-C₆ organic alkoxysilanes.

Particularly preferably, the targeted hydrolysis is carried out by adding a substoichiometric amount of water. In this case, the amount of water used is below the amount that would theoretically be required to hydrolyze all the hydrolysable C₁-C₆ alkoxy groups present on the Si atoms, i.e., the alkoxysilane groups. Partial hydrolysis of the organic C₁-C₆ alkoxy silanes is therefore particularly preferred.

The stoichiometric ratio of water to the organic C₁-C₆ alkoxy silanes can be defined by the amount of substance equivalent water (S-W), these are calculated according to the following formula:

$$S - W = \frac{mol ( Water )}{mol ( Silane ) \times n ( Alkoxy )}$$

with
S-W=Mass equivalent water
mol(water)=molar quantity of water used
mol(silanes)=total molar amount of C₁-C₆ alkoxy silanes used in the reaction
n(Alkoxy)=Number of C₁-C₆ alkoxy groups per C₁-C₆ alkoxy silane

In other words, the molar equivalent of water is the molar ratio of the molar amount of water used to the total molar number of hydrolysable C₁-C₆ alkoxy groups present on the C₁-C₆ alkoxysilanes used.

In another particularly preferred embodiment, a process as contemplated herein is exemplified in that the organic C₁-C₆-alkoxysilane or organic C₁-C₆-alkoxysilanes are selectively hydrolyzed by addition of about 0.10 to about 0.80 molar equivalents of water (S-W), preferably of about 0.15 to about 0.70, more preferably of about 0.20 to about 0.60 and most preferably of about 0.25 to about 0.50 molar equivalents of water, the molar equivalents of water being calculated according to the formula

$$S - W = \frac{mol ( Water )}{mol ( Silane ) \times n ( Alkoxy )}$$

with
S-W=Mass equivalent water
mol(water)=molar quantity of water used
mol(silanes)=total molar amount of C₁-C₆ alkoxy silanes used in the reaction
n(Alkoxy)=Number of C₁-C₆ alkoxy groups per C₁-C₆ alkoxy silane.

If the mixture of organic C₁-C₆ alkoxy siloxanes is prepared by the catalyzed targeted hydrolysis of a C₁-C₆ alkoxy silane, the number of C₁-C₆ alkoxy groups per C₁-C₆ alkoxy silane is equal to the number of C₁-C₆ alkoxy groups present in the silane of that structure. However, if a mixture of structurally different C₁-C₆ alkoxy silanes is used in the hydrolysis, n(alkoxy) corresponds to the number average of the C₁-C₆ alkoxy groups of the C₁-C₆ alkoxy silanes. Using the formula S-W, the substance equivalents of water are then calculated as follows, for example:

$$S - W = \frac{mol ( Water )}{[ mol ( Silane ( I ) ) \times n ( Alkoxy ( I ) ) ] + [ mol ( Silane ( IV ) ) \times n ( Alkoxy ( IV ) ) ]}$$

with
S-W=Mass equivalent water
mol(water)=molar quantity of water used
mol(silane (I))=total molar amount of C₁-C₆ alkoxy silanes of the formula (I) used
n(alkoxy (I))=number of C₁-C₆ alkoxy groups per C₁-C₆ alkoxy silane of the formula (I)
mol(silane (IV))=total molar amount of C₁-C₆ alkoxy silanes of the formula (IV) used
n(alkoxy (IV))=number of C₁-C₆ alkoxy groups per C₁-C₆ alkoxy silane of the formula (IV)

### Example

In a reaction vessel, 20.0 g of 3-aminopropyltriethoxsilane (C₉H₂₃NO₃Si=221.37 g/mol) and 50.0 g of methyltrimethoxysilane (C₄H₁₂O₃Si=136.22 g/mol) were mixed together. 25 g of ethanol was added to this mixture with stirring.

20.0 g 3-aminopropyltriethoxsilane=0.0903 mol (3 hydrolysable alkoxy groups per molecule)
50.0 g methyltrimethoxysilane=0.367 mol (3 hydrolysable alkoxy groups per molecule)

Then, 10.0 g of water (18.015 g/mol) provided with catalyst was added with stirring.

10.0 g water=0.555 mol

Mass equivalent water=0.555 mol/[1(3×0.090 mol)+(3×0.367 mol)]=0.40

In this reaction, the C₁-C₆ alkoxysilanes used were reacted with 0.40 molar equivalents of water.

If other or different mixtures of C₁-C₆ alkoxy silanes are used, the formula S-W is adapted accordingly.

The reaction of the organic C₁-C₆ alkoxy silanes with water can take place in different ways. The reaction starts as soon as the C₁-C₆ alkoxy silanes come into contact with water by mixing. As soon as C₁-C₆ alkoxy silanes and water come into contact, an exothermic hydrolysis reaction takes place according to the following scheme (reaction scheme using the example of 3-aminopropyltriethoxysilane):

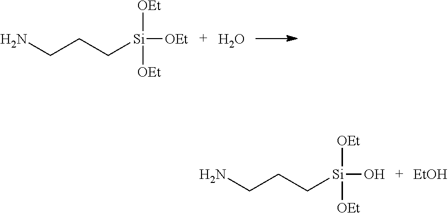

Depending on the number of hydrolysable C₁-C₆ alkoxy groups per silane molecule, the hydrolysis reaction can also occur several times per C₁-C₆ alkoxy silane used:

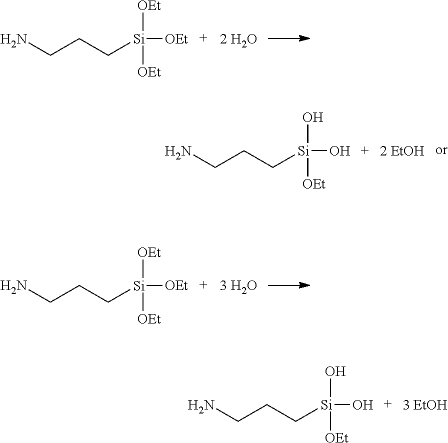

Hydrolysis using the example of methyltrimethoxysilane:

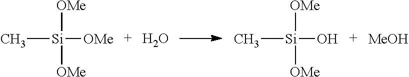

Depending on the amount of water used, the hydrolysis reaction can also take place several times per C₁-C₆ alkoxy silane used:

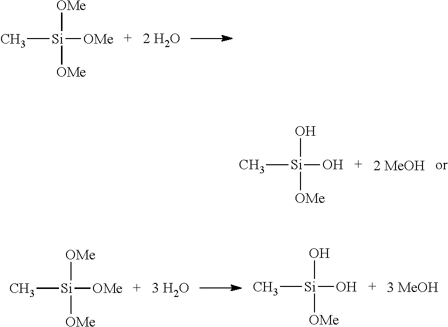

### Precondensation

Following the hydrolysis or quasi simultaneously with the hydrolysis, condensation of the partially (or in parts completely) hydrolyzed C₁-C₆ alkoxy silanes takes place. The precondensation can proceed, for example, according to the following scheme:

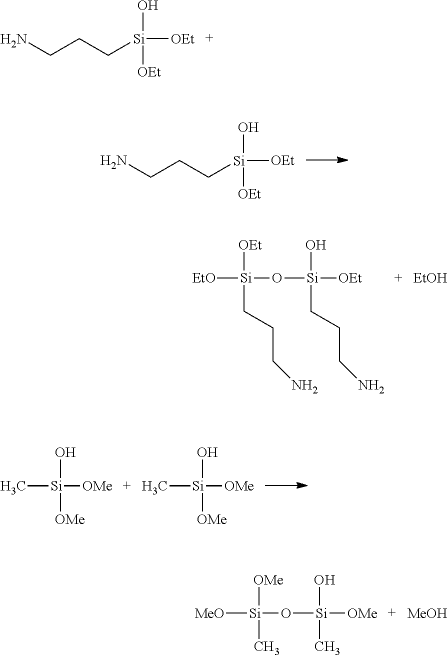

Both partially hydrolyzed and fully hydrolyzed C₁-C₆ alkoxysilanes can participate in the condensation reaction, undergoing condensation with not yet reacted, partially or also fully hydrolyzed C₁-C₆ alkoxysilanes.

Possible condensation reactions include (shown using the mixture (3-aminopropyl)triethoxysilane and methyltrimethoxysilane):

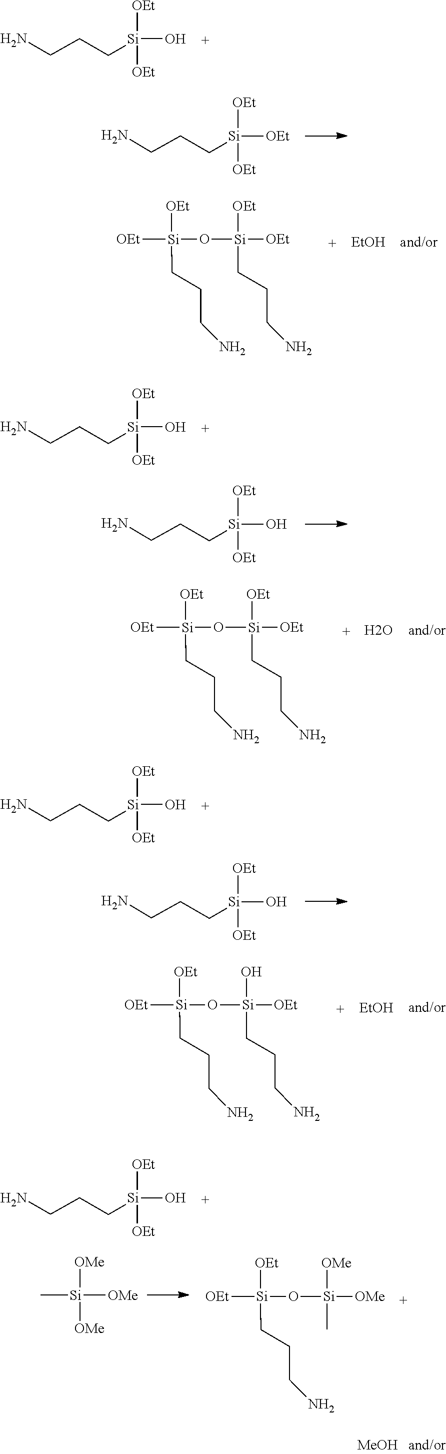

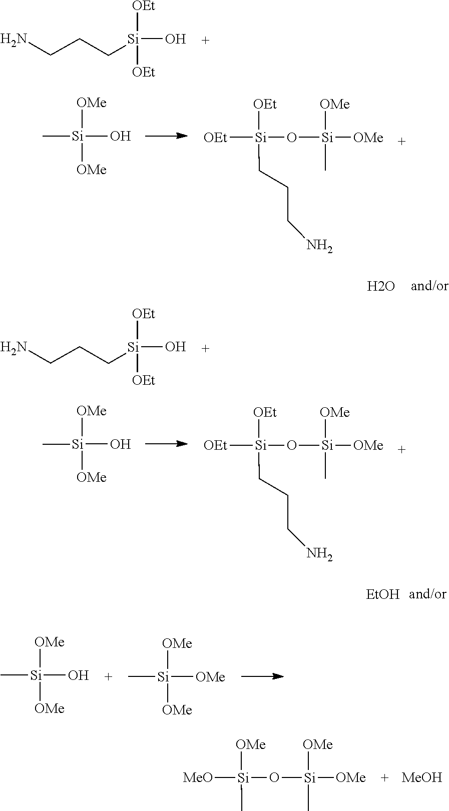

In the above exemplary reaction schemes the condensation to a dimer is shown in each case, but further condensations to oligomers with several silane atoms are also possible and also preferred.

Therefore, for the purposes of the present disclosure, a precondensation is understood to be the condensation of the organic C₁-C₆ alkoxysilanes to at least one dimer.

In other words, the first object of the present disclosure is a process for the treatment of keratinous material, in particular human hair, wherein a cosmetic agent is applied to the keratinous material and is rinsed off again after a contact time, exemplified in that the cosmetic agent contains a mixture of organic C₁-C₆-alkoxy-siloxanes, which is obtained by mixing one or more organic C₁-C₆-alkoxy-silanes with a solvent other than water and hydrolyzing and condensing them by adding water, preferably by adding a substoichiometric amount of water, and catalyst.

Or, in simplified terms, the first object of the present disclosure is a process for the treatment of keratinous material, in particular human hair, wherein a cosmetic agent is applied to the keratinous material and rinsed off again after a contact time, exemplified in that the cosmetic agent contains a mixture of organic C₁-C₆ alkoxy-siloxanes, which is obtained by mixing one or more organic C₁-C₆ alkoxysilanes with a solvent other than water and then adding water and catalyst to this mixture.

For example, both partially hydrolyzed and fully hydrolyzed C₁-C₆ alkoxysilanes of the formula (I) can participate in these condensation reactions, which undergo condensation with not yet reacted, partially or also fully hydrolyzed C₁-C₆ alkoxysilanes of the formula (I). In this case, the C₁-C₆ alkoxysilanes of formula (I) react with themselves.

Furthermore, both partially hydrolyzed and fully hydrolyzed C₁-C₆-alkoxysilanes of the formula (I) can also participate in the condensation reactions, which undergo condensation with as yet unreacted, partially or also fully hydrolyzed C₁-C₁₂-alkyl-C₁-C₆-alkoxy silanes of the formula (IV). In this case, the C₁-C₆ alkoxysilanes of formula (I) react with the C₁-C₆ alkoxysilanes of formula (IV).

Furthermore, both partially hydrolyzed and fully hydrolyzed C₁-C₆ alkoxy silanes of the formula (IV) can also participate in the condensation reactions, which undergo condensation with not yet reacted, partially or also fully hydrolyzed C₁-C₆ alkoxy silanes of the formula (IV). In this case, the C₁-C₆ alkoxy silanes of formula (IV) react with themselves.

In the exemplary reaction scheme above, condensation to a dimer is shown, but condensation to oligomers with multiple silane atoms is also possible and also preferred.

The extent of the condensation reaction is partly determined by the amount of water added. Preferably, the amount of water is such that the precondensation is a partial condensation, where “partial condensation” or “partial condensation” in this context means that not all of the condensable groups of the silanes presented react with each other, so that the resulting organic silicon compound still has on average at least one hydrolysable/condensable group per molecule in each case.

### Catalyst

In the preparation of the organic C₁-C₆ alkoxy siloxane(s), the organic C₁-C₆ alkoxysilane(s) is (are) mixed with a solvent other than water and selectively hydrolyzed and precondensed by the addition of water and a catalyst

The addition of the catalyst initiates or accelerates the hydrolysis reaction.

Furthermore, it is believed that the addition of the catalyst also affects the condensation reaction in a way that causes condensation to occur to a significant extent even without subsequent distillation off of the solvents. Accordingly, the addition of the catalyst can presumably avoid subsequent distillation off of the solvents, thus taking advantage of the previously described benefits of the solvent components remaining in the siloxane mixture.

By a catalyst, the skilled person understands a substance that increases the reaction rate by lowering the activation energy of a chemical reaction without itself being consumed.

The catalyst can be added before or after the water is added.

For the preparation of mixtures of organic C₁-C₆ alkoxy siloxanes, it has proved particularly advantageous to use a catalyst that can be dissolved or dispersed in water and is then added to the mixture of organic C₁-C₆ alkoxy silanes and solvent together with the water as a solution or dispersion.

Very preferably, the catalyst is selected from the group of inorganic or organic acids and inorganic or organic bases.

Particularly well-suited catalysts are inorganic and organic acids, which can preferably be selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, and 1-hydroxyethane-1,1-diphosphonic acid. Explicitly, sulfuric acid, hydrochloric acid and maleic acid are particularly preferred.

In another very particularly preferred embodiment, a process as contemplated herein is exemplified in that the catalyst is selected from the group of inorganic and organic acids, preferably from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid and 1-hydroxyethane-1,1-diphosphonic acid.

Other particularly suitable catalysts are inorganic and organic bases, which can preferably be selected from the group of sodium hydroxide, potassium hydroxide, magnesium hydroxide and calcium hydroxide. Sodium hydroxide and potassium hydroxide are particularly preferred.

Other bases that can be used include ammonia, alkanolamines and/or basic amino acids.

Alkanolamines may be selected from primary amines having a C₂-C₆ alkyl parent bearing at least one hydroxyl group. Preferred alkanolamines are selected from the group formed by 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

For the purposes of the present disclosure, an amino acid is an organic compound containing at least one protonatable amino group and at least one —COOH or —SO₃H group in its structure. Preferred amino acids are amino carboxylic acids, especially α-(alpha)-amino carboxylic acids and ω-amino carboxylic acids, whereby α-amino carboxylic acids are particularly preferred.

As contemplated herein, basic amino acids are those amino acids which have an isoelectric point pI of greater than about 7.0.

Basic α-amino carboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, an agent as contemplated herein is therefore exemplified in that the alkalizing agent is a basic amino acid from the group arginine, lysine, ornithine and/or histidine.

In addition, other inorganic alkalizing agents or bases can also be used. Inorganic alkalizing agents that can be used as contemplated herein can be selected, for example, from the group formed by sodium phosphate, potassium phosphate, sodium silicate, sodium metasilicate, potassium silicate, sodium carbonate and potassium carbonate.

In another very particularly preferred embodiment, a process as contemplated herein is exemplified in that the catalyst is selected from the group of inorganic and organic bases, preferably from the group of sodium hydroxide, potassium hydroxide, magnesium hydroxide and calcium hydroxide.

As contemplated herein, the catalysts are preferably used in the usual quantity ranges for catalysts. Since the catalysts accelerate the hydrolysis or condensation without being consumed themselves, the quantities used can be chosen to be correspondingly low.

Thus, the catalyst or catalysts can be used in an amount range from about 0.0000001 to about 2.0 wt. %, preferably from about 0.0001 to about 1.5 wt. %, and most preferably from about 0.01 to about 1.0 wt. %. Here, the figure in wt. % refers to the total amount of catalysts used in relation to the total amount of organic C₁-C₆ alkoxy siloxanes plus solvent plus water.

Preparation Process of the Mixture of Organic C₁-C₆ Alkoxy Siloxanes

In principle, various methods are conceivable for preparing the mixture of organic C₁-C₆ alkoxy siloxanes.

For example, one possible manufacturing process is as follows:

i) A quantity of solvent, for example ethanol or methanol, and a quantity of organic C₁-C₆ alkoxysilane, for example methyltrimethoxysilane or methyltriethoxysilane, are placed in a round bottom flask.

ii) The filled round bottom flask is equipped with a stirrer and a thermometer.

iii) Then the round bottom flask is clamped into a stirring apparatus and connected to the cooling system.

iv) The flask contents are brought to the desired temperature by employing an oil bath while stirring at 500 rpm.

v) When the desired temperature is reached, the amount of water with catalyst is dosed into the round bottom flask using a 100 ml dropping funnel over 3 minutes. The dropping funnel is removed from the apparatus and replaced by a new dropping funnel containing the previously calculated amount of another organic C₁-C₆ alkoxysilane, for example (3-aminopropyl)-triethoxysilane.

vi.) 10 to 60 minutes after completion of the addition of water plus catalyst, the second organic C₁-C₆ alkoxysilane is added.

vii.) It is stirred for another 30 to 240 minutes.

viii) The mixture of organic C₁-C₆-alkoxy-siloxanes prepared in this way is filled into a tight container while still hot.

This production process is particularly well suited when at least one acid, for example an acid selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, and 1-hydroxyethane-1,1-diphosphonic acid, is used as the catalyst.

Furthermore, this production process is also particularly suitable if at least one base, preferably from the group of sodium hydroxide, potassium hydroxide, magnesium hydroxide and calcium hydroxide, is used as catalyst.

As this production process shows, it is as contemplated herein and, in the case of the use of acids or bases as catalysts, even particularly preferred if the mixing of a first organic C₁-C₆-alkoxysilane with the solvent and the addition of water and catalyst, which initiate the hydrolysis and precondensation of the first C₁-C₆-alkoxysilane, are followed additionally by the addition of a second organic C₁-C₆-alkoxysilane.

Mixtures of organic C₁-C₆ alkoxy-siloxanes with particularly good cosmetic properties were obtained, in particular, when at least one organic C₁-C₆ alkoxysilane of formula (IV) was used in step i.) of the above process, and when at least one other organic C₁-C₆ alkoxysilane of formula (I) was added in steps v.) or vi.) of the process.

In another very particularly preferred embodiment, a process as contemplated herein is exemplified in that the cosmetic agent comprises a mixture of organic C₁-C₆ alkoxy-siloxanes obtained by

(1) one or more organic C₁-C₆ alkoxysilanes of the formula (IV) are mixed with the solvent other than water and selectively hydrolyzed and precondensed by addition of water and catalyst,

(2) the mixture obtained in step (1) is stirred for a period of from about 5 minutes to about 3 hours, preferably from about 10 minutes to about 40 minutes, at a temperature of from about 30 to about 80° C., preferably from about 40 to about 70° C., and then

(3) the mixture obtained in step (2) is mixed with one or more organic C₁-C₆ alkoxysilanes of formula (I).

In other words, a preferred process as contemplated herein is exemplified in that the cosmetic agent comprises a mixture of organic C₁-C₆ alkoxy-siloxanes obtained by

(1) one or more organic C₁-C₆ alkoxysilanes of formula (IV) are mixed with the solvent other than water and water and catalyst are added to this mixture,

(2) the mixture obtained in step (1) is stirred for a period of from about 5 minutes to about 3 hours, preferably from about 10 minutes to about 40 minutes, at a temperature of from about 30 to about 80° C., preferably from about 40 to about 70° C., and then

(3) the mixture obtained in step (2) is mixed with one or more organic C₁-C₆ alkoxysilanes of formula (I).

In a further very particularly preferred embodiment, a process as contemplated herein is exemplified in that

(4) the mixture obtained in step (3) is stirred at a temperature of from about 30 to about 80° C., preferably from about 40 to about 70° C., for a period of from about 20 minutes to about 24 hours, preferably from about 40 minutes to about 6 hours.

Another possible manufacturing process is the following:

i.) A quantity of solvent, for example ethanol or methanol, and a quantity of organic C₁-C₆ alkoxysilanes, for example methyltrimethoxysilane and/or methyltriethoxysilane and/or (3-aminopropyl)-triethoxysilane, are placed in a round bottom flask.

Particularly preferred in this step are a mixture of methyltrimethoxysilane and (3-aminopropyl)-triethoxysilane, a mixture of methyltriethoxysilane and (3-aminopropyl)-triethoxysilane, or a mixture of ethyltriethoxysilane and (3-aminopropyl)-triethoxysilane.

ii.) The filled round bottom flask is equipped with a stirrer and a thermometer.

iii.) Then the round-bottom flask is clamped into a stirring apparatus and connected to the cooling system.

iv.) The contents of the flask are brought to the desired temperature by employing an oil bath while stirring at about 500 rpm.

iv.) When the desired temperature is reached, the amount of water with catalyst is dosed into the round bottom flask using a 100 ml dropping funnel over about 3 minutes.

vi.) It is stirred for about another 30 to 240 minutes.

vii.) The mixture of organic C₁-C₆ alkoxy siloxanes produced in this way is filled into a tight container while still hot.

This production process is particularly well suited when at least one base, for example a base selected from the group of sodium hydroxide, potassium hydroxide, magnesium hydroxide and calcium hydroxide, is used as the catalyst.

Mixtures of organic C₁-C₆ alkoxy-siloxanes with particularly good cosmetic properties were obtained in particular when at least one organic C₁-C₆ alkoxysilane of the formula (IV) and additionally at least one further organic C₁-C₆ alkoxysilane of the formula (I) were used in step i.) of the above-mentioned process.

In another very particularly preferred embodiment, a process as contemplated herein is exemplified in that the cosmetic agent comprises a mixture of organic C₁-C₆ alkoxy-siloxanes obtained by

(1) one or more organic C₁-C₆-alkoxysilanes of the formula (I) are mixed with one or more organic C₁-C₆-alkoxysilanes of the formula (IV) and the solvent other than water and are selectively hydrolyzed and precondensed by addition of water and catalyst.

In other words, a preferred process as contemplated herein is exemplified in that the cosmetic agent comprises a mixture of organic C₁-C₆ alkoxy-siloxanes obtained by

(1) one or more organic C₁-C₆ alkoxysilanes of the formula (I) are mixed with one or more organic C₁-C₆ alkoxysilanes of the formula (IV) and the solvent other than water, and water and catalyst are added to this mixture.

In a further very particularly preferred embodiment, the aforementioned process as contemplated herein is exemplified in that

(2) the mixture obtained in step (1) is stirred at a temperature of from about 30 to about 80° C., preferably from about 40 to about 70° C., for a period of from about 20 hours to about 3 days, preferably from about 2 to about 24 hours.

The mixtures of organic C₁-C₆ alkoxy siloxanes prepared in this way can alternatively be referred to as silane blends.

The organic C₁-C₆ alkoxysilanes used to prepare the mixture are partially hydrolyzed and condensed, so that dimeric, trimeric, oligomeric and, in small parts, probably also polymeric C₁-C₆ alkoxy siloxanes with higher molecular weight are present in the mixture. Since not all of the C₁-C₆ alkoxy groups bonded to the silicone atoms have reacted off, the mixture still has reactive functional groups that react off only during subsequent application to the keratin material and can thus condense further to form even higher polymer systems. When these mixtures are used in cosmetic agents for the treatment of keratinous material, very durable and uniform film or coatings can be produced on the keratinous material in this way.

### Process for the Treatment of Keratinous Material

Agents applied to keratin materials, especially human hair, are usually agents with a high water content. The cosmetic agent used in the process of the first object of the present disclosure also represents an agent ready for use which preferably has a high water content, i.e. a water content of more than about 50 wt. %, preferably more than about 60 wt. % and particularly preferably more than about 70 wt. %.

Since the previously described mixture of organic C₁-C₆ alkoxy siloxanes still comprises reactive groups and can be hydrolyzed by an excess of water during longer storage times, it is preferably provided to the user in the form of a low-water concentrate and mixed with a water-containing carrier only shortly before use and diluted in this way.

A second object of the present application is therefore a process for treating keratinous material, in particular human hair, comprising the following steps in the order indicated:

(i) Providing a mixture of organic C₁-C₆ alkoxy siloxanes, the preparation of which was disclosed in detail in the description of the first subject of the present disclosure,

(ii) Blending the mixture of C₁-C₆ organic alkoxy siloxanes with a water-containing cosmetic carrier to obtain a ready-to-use cosmetic agent,

(iii) applying the ready-to-use cosmetic agent prepared in step (ii) to the keratinous material,

(iv) Exposure of the agent applied in step (iii) to the keratinous material,

(v) rinse off the agent from the keratinous material,

(vi) if necessary, applying a post-treatment agent to the keratinous material,

(vii) where appropriate, exposure of the keratinous material to the post-treatment agent; and

(viii) if necessary, rinsing the post-treatment agent off the keratinous material.

In step (i) of the process, the mixture of C₁-C₆-organic alkoxy siloxanes is provided. This can be done, for example, in the form of a separately prepared blend or concentrate, which is preferably packaged in an airtight manner. Just prior to application, in step (ii), the user or hairdresser may mix this concentrate with a water-based cosmetic carrier to obtain a ready-to-use cosmetic agent.

For reasons of storage stability, the mixture of organic C₁-C₆ alkoxy siloxanes preferably contains no other cosmetic ingredients. However, the aqueous cosmetic carrier may contain various other ingredients.

The cosmetic ingredients that can be used optionally in the cosmetic carrier may be any suitable ingredients to impart further positive properties to the agent. For example, cosmetic ingredients from the group of thickening or film-forming polymers, surface-active compounds from the group of nonionic, cationic, anionic or zwitterionic/amphoteric surfactants, coloring compounds from the group of pigments, direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

Mixing of the C₁-C₆ alkoxy-siloxane mixture and the water-containing cosmetic carrier can be achieved, for example, by stirring or shaking. It is particularly advantageous to prepare the two preparations separately in two containers and then, prior to use, to transfer the entire amount of the C₁-C₆ alkoxy-siloxane mixture from their container to the container containing the aqueous cosmetic carrier.

The C₁-C₆ alkoxy-siloxane mixture and the aqueous cosmetic carrier can be mixed together in different proportions.

Particularly preferably, the C₁-C₆ alkoxy-siloxane mixture is used in the form of a relatively highly concentrated, low-water silane blend, which is virtually diluted by mixing with the water-containing cosmetic carrier. For this reason, it is particularly preferred to blend the C₁-C₆ alkoxy-siloxane mixture with an excess weight of cosmetic carrier. For example, about 1 part by weight of siloxane mixture can be mixed with about 20 parts by weight of carrier, or about 1 part by weight of siloxane mixture is mixed with about 10 parts by weight of carrier, or about 1 part by weight of siloxane mixture is mixed with about 5 parts by weight of carrier.

In step (iii) of the process, the ready-to-use cosmetic agent prepared in step (ii) is applied to the keratinous material, in particular to human hair. The application can be done with the help of the gloved hand or with the help of a brush, a spout or an applicator.

Then, in step (iv), the applied agent is allowed to act into or onto the keratinous material. Suitable exposure times here are from about 30 seconds to about 60 minutes, preferably from about 1 to about 30 minutes, further preferably from about 1 to about 20 minutes, and most preferably from about 1 to about 10 minutes.

Then, in step (v), the agent is rinsed off the keratinous material, or hair. Rinsing is preferably done with tap water only.

In steps (vi), (vii) and (viii), a post-treatment agent can still optionally be applied to the keratinous material, allowed to act and then rinsed out again if necessary.

It is quite preferred if the keratin treatment process described above is a process for dyeing human hair. In this embodiment, the ready-to-use cosmetic agent applied in step (ii) specially preferably additionally contains at least pigment and/or a direct dye.

In another very particularly preferred embodiment, a process as contemplated herein is exemplified in that it is a process for coloring human hair and in that the ready-to-use cosmetic agent applied in step (iii) additionally contains at least one pigment and/or a direct dye.

The use of a post-treatment agent may also be preferred, in particular, if the process for treating keratinous material is a dyeing process in which a coloring compound, such as in particular in pigment, is still to be applied to the keratinous materials in a downstream step.

In another very particularly preferred embodiment, a process as contemplated herein is exemplified in that it is a process for dyeing hair and the after treatment agent applied in step (vi) comprises at least one pigment and/or a direct dye.

Pigment and/or a Direct Colorant

In the course of the work leading to the present disclosure, it was observed that the films formed on the keratin material possessed not only good rub fastness but also particularly high color intensity when a coloring compound from the group of pigments and/or direct dyes was used in the process. The use of pigments has proved to be particularly preferable.

The colorant compound(s) may be selected from the group of pigments and direct dyes, where direct dyes may also be photochromic dyes and thermochromic dyes.

Pigments within the meaning of the present disclosure are coloring compounds which have a solubility in water at 25° C. of less than 0.5 g/L, preferably less than 0.1 g/L, even more preferably less than 0.05 g/L. Water solubility can be determined, for example, by the method described below: 0.5 g of the pigment are weighed in a beaker. A beaker glass is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable color pigments can be of inorganic and/or organic origin.

In a preferred embodiment, an agent used in the process as contemplated herein is exemplified in that it contains at least one coloring compound from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulfates, chromates and/or molybdates. In particular, preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred colorants from the group of pigments as contemplated herein. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

In a very particularly preferred embodiment, a process as contemplated herein is exemplified in that the corresponding agent contains at least one colorant compound from the group of inorganic pigments selected from the group of colored metal oxides, metal hydroxides, metal oxide hydrates, silicates, metal sulfides, complex metal cyanides, metal sulfates, bronze pigments and/or from colored mica- or mica-based pigments coated with at least one metal oxide and/or a metal oxychloride.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, a process as contemplated herein is exemplified in that the corresponding agent contains at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a process as contemplated herein is exemplified in that the corresponding agent contains at least one colorant compound selected from mica- or mica-based pigments reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck®, Ariabel® and Unipure® from Sensient®, Prestige® from Eckart® Cosmetic Colors and Sunshine® from Sunstar®.

Particularly preferred color pigments with the trade name Colorona® are, for example:

### Colorona® Copper, Merck®, MICA, CI 77491 (IRON OXIDES)

### Colorona® Passion Orange, Merck®, Mica, CI 77491 (Iron Oxides), Alumina

### Colorona® Patina Silver, Merck®, MICA, CI 77499 (IRON OXIDES), CI 77891 (TITANIUM DIOXIDE)

### Colorona® RY, Merck®, CI 77891 (TITANIUM DIOXIDE), MICA, CI 75470 (CARMINE)

### Colorona® Oriental Beige, Merck®, MICA, CI 77891 (TITANIUM DIOXIDE), CI 77491 (IRON OXIDES)

### Colorona® Dark Blue, Merck®, MICA, TITANIUM DIOXIDE, FERRIC FERROCYANIDE

### Colorona® Chameleon, Merck®, CI 77491 (IRON OXIDES), MICA

### Colorona® Aborigine Amber, Merck®, MICA, CI 77499 (IRON OXIDES), CI 77891 (TITANIUM DIOXIDE)

### Colorona® Blackstar Blue, Merck®, CI 77499 (IRON OXIDES), MICA

### Colorona® Patagonian Purple, Merck®, MICA, CI 77491 (IRON OXIDES), CI 77891 (TITANIUM DIOXIDE), CI 77510 (FERRIC FERROCYANIDE)

### Colorona® Red Brown, Merck®, MICA, CI 77491 (IRON OXIDES), CI 77891 (TITANIUM DIOXIDE)

### Colorona® Russet, Merck®, CI 77491 (TITANIUM DIOXIDE), MICA, CI 77891 (IRON OXIDES)

### Colorona® Imperial Red, Merck®, MICA, TITANIUM DIOXIDE (CI 77891), D&C RED NO. 30 (CI 73360)

### Colorona® Majestic Green, Merck®, CI 77891 (TITANIUM DIOXIDE), MICA, CI 77288 (CHROMIUM OXIDE GREENS)

### Colorona® Light Blue, Merck®, MICA, TITANIUM DIOXIDE (CI 77891), FERRIC FERROCYANIDE (CI 77510)

### Colorona® Red Gold, Merck®, MICA, CI 77891 (TITANIUM DIOXIDE), CI 77491 (IRON OXIDES)

### Colorona® Gold Plus MP 25, Merck®, MICA, TITANIUM DIOXIDE (CI 77891), IRON OXIDES (CI 77491)

### Colorona® Carmine Red, Merck®, MICA, TITANIUM DIOXIDE, CARMINE

### Colorona® Blackstar Green, Merck®, MICA, CI 77499 (IRON OXIDES)

### Colorona® Bordeaux, Merck®, MICA, CI 77491 (IRON OXIDES)

### Colorona® Bronze, Merck®, MICA, CI 77491 (IRON OXIDES)

### Colorona® Bronze Fine, Merck®, MICA, CI 77491 (IRON OXIDES)

### Colorona® Fine Gold MP 20, Merck®, MICA, CI 77891 (TITANIUM DIOXIDE), CI 77491 (IRON OXIDES)

### Colorona® Sienna Fine, Merck®, CI 77491 (IRON OXIDES), MICA

### Colorona® Sienna, Merck®, MICA, CI 77491 (IRON OXIDES)

Colorona® Precious Gold, Merck®, Mica, CI 77891 (Titanium dioxide), Silica, CI 77491 (Iron oxides), Tin oxide

### Colorona® Sun Gold Sparkle MP 29, Merck®, MICA, TITANIUM DIOXIDE, IRON OXIDES, MICA, CI 77891, CI 77491 (EU)

Colorona® Mica Black, Merck®, CI 77499 (Iron oxides), Mica, CI 77891 (Titanium dioxide)
Colorona® Bright Gold, Merck®, Mica, CI 77891 (Titanium dioxide), CI 77491 (Iron oxides)

### Colorona® Blackstar Gold, Merck®, MICA, CI 77499 (IRON OXIDES)

Other particularly preferred color pigments with the trade name Xirona® are for example:

### Xirona® Golden Sky, Merck®, Silica, CI 77891 (Titanium Dioxide), Tin Oxide

### Xirona® Caribbean Blue, Merck®, Mica, CI 77891 (Titanium Dioxide), Silica, Tin Oxide

### Xirona® Kiwi Rose, Merck®, Silica, CI 77891 (Titanium Dioxide), Tin Oxide

### Xirona® Magic Mauve, Merck®, Silica, CI 77891 (Titanium Dioxide), Tin Oxide.

In addition, particularly preferred color pigments with the trade name Unipure® are for example:

### Unipure® Red LC 381 EM, Sensient® CI 77491 (Iron Oxides), Silica

### Unipure® Black LC 989 EM, Sensient®, CI 77499 (Iron Oxides), Silica

### Unipure® Yellow LC 182 EM, Sensient®, CI 77492 (Iron Oxides), Silica

Timiron® Synwhite Satin, Merck®, Synthetic Fluorphlogopite, Titanium Dioxide, Tin Oxide

### Timiron® Super Blue, Merck®, Mica, CI 77891 (Titanium Dioxide)

### Timiron® Diamond Cluster MP 149, Merck®, Mica, CI 77891 (Titanium Dioxide)

Timiron® Splendid Gold, Merck®, CI 77891 (titanium dioxide), mica, silicon dioxide

### Timiron® Super Sulver, Merck®, Mica, CI 77891 (Titanium Dioxide)

In a further embodiment, the agent used in the process as contemplated herein may also contain one or more coloring compounds from the group of organic pigments

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, a process as contemplated herein is exemplified in that the corresponding agent contains at least one coloring compound from the group of organic pigments selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lacquer. In the sense of the present disclosure, the term color lacquer means particles comprising a layer of absorbed dyes, the unit of particle and dye being insoluble under the above mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the aforementioned pigments as contemplated herein is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D₅₀ of about 1.0 to about 50 μm, preferably about 5.0 to about 45 μm, preferably about 10 to about 40 μm, in particular about 14 to about 30 μm. The average particle size D₅₀, for example, can be determined using dynamic light scattering (DLS).

Pigments with a specific shape may also be used to color the keratin material. For example, a pigment based on a lamellar and/or a lenticular substrate platelet can be used. Furthermore, coloring based on a substrate platelet comprising a vacuum metallized pigment is also possible.

In a further embodiment, a process as contemplated herein may be exemplified in that the corresponding agent also comprises one or more colorant compounds selected from the group of lamellar substrate platelet-based pigments, lenticular substrate platelet-based pigments and vacuum metallized pigments.

The substrate platelets of this type have an average thickness of at most about 50 nm, preferably less than about 30 nm, particularly preferably at most about 25 nm, for example at most about 20 nm. The average thickness of the substrate platelets is at least about 1 nm, preferably at least about 2.5 nm, particularly preferably at least about 5 nm, for example at least about 10 nm. Preferred ranges for substrate platelet thickness are about 2.5 to about 50 nm, about 5 to about 50 nm, about 10 to about 50 nm; about 2.5 to about 30 nm, about 5 to about 30 nm, about 10 to about 30 nm; about 2.5 to about 25 nm, about 5 to about 25 nm, about 10 to about 25 nm, about 2.5 to about 20 nm, about 5 to about 20 nm, and about 10 to about 20 nm. Preferably, each substrate plate has a thickness that is as uniform as possible.

Due to the low thickness of the substrate platelets, the pigment exhibits particularly high hiding power.

The substrate platelets have a monolithic structure. Monolithic in this context means having a single closed unit without fractures, stratifications or inclusions, although structural changes may occur within the substrate platelets. The substrate platelets are preferably homogeneously structured, i.e. there is no concentration gradient within the platelets. In particular, the substrate platelets do not have a layered structure and do not have any particles or particles distributed in them.

The size of the substrate platelet can be adjusted to the respective application purpose, especially the desired effect on the keratinic material. Typically, the substrate platelets have an average largest diameter of about 2 to about 200 μm, especially about 5 to about 100 μm.

In a preferred design, the aspect ratio, expressed by the ratio of the average size to the average thickness, is at least about 80, preferably at least about 200, more preferably at least about 500, more preferably more than about 750. The average size of the uncoated substrate platelets is the d50 value of the uncoated substrate platelets. Unless otherwise stated, the d50 value was determined using a Sympatec Helos device with quixel wet dispersion. To prepare the sample, the sample to be analyzed was pre-dispersed in isopropanol for about 3 minutes.

The substrate platelets can be composed of any material that can be formed into platelet shape.

They can be of natural origin, but also synthetically produced. Materials from which the substrate platelets can be constructed include metals and metal alloys, metal oxides, preferably aluminum oxide, inorganic compounds and minerals such as mica and (semi-)precious stones, and plastics. Preferably, the substrate platelets are constructed of metal (alloy).

Any metal suitable for metallic luster pigments can be used. Such metals include iron and steel, as well as all air and water resistant (semi)metals such as platinum, zinc, chromium, molybdenum and silicon, and their alloys such as aluminum bronzes and brass. Preferred metals are aluminum, copper, silver and gold. Preferred substrate platelets include aluminum platelets and brass platelets, with aluminum substrate platelets being particularly preferred.

Lamellar substrate platelets are exemplified by an irregularly structured edge and are also referred to as “cornflakes” due to their appearance.

Due to their irregular structure, pigments based on lamellar substrate platelets generate a high proportion of scattered light. In addition, pigments based on lamellar substrate platelets do not completely cover the existing color of a keratinous material, and effects analogous to natural graying can be achieved, for example.

Lenticular (=lens-shaped) substrate platelets have an essentially regular round edge and are also called “silver dollars” due to their appearance. Due to their regular structure, the proportion of reflected light predominates in pigments based on lenticular substrate platelets.

Vacuum metallized pigments (VMP) can be obtained, for example, by releasing metals, metal alloys or metal oxides from suitably coated films. They are exemplified by a particularly low thickness of the substrate platelets in the range of about 5 to about 50 nm and a particularly smooth surface with increased reflectivity. Substrate platelets comprising a vacuum metallized pigment are also referred to as VMP substrate platelets in the context of this application. VMP substrate platelets of aluminum can be obtained, for example, by releasing aluminum from metallized films.

The metal or metal alloy substrate platelets can be passivated, for example by anodizing (oxide layer) or chromating.

Uncoated lamellar, lenticular and/or VPM substrate platelets, especially those made of metal or metal alloy, reflect the incident light to a high degree and create a light-dark flop but no color impression.

A color impression can be created by optical interference effects, for example. Such pigments can be based on at least single-coated substrate platelets. These show interference effects by superimposing differently refracted and reflected light beams.

Accordingly, preferred pigments are pigments based on a coated lamellar substrate platelet. The substrate platelet preferably has at least one coating B of a highly refractive metal oxide having a coating thickness of at least about 50 nm. There is preferably another coating A between the coating B and the surface of the substrate platelet. If necessary, there is a further coating C on the layer B, which is different from the layer B underneath.

Suitable materials for coatings A, B and C are all substances that can be applied to the substrate platelets in a film-like and permanent manner and, in the case of layer A and B, have the required optical properties. Generally, coating part of the surface of the substrate platelets is sufficient to obtain a pigment with a glossy effect. For example, only the top and/or bottom of the substrate platelets may be coated, with the side surface(s) omitted. Preferably, the entire surface of the optionally passivated substrate platelets, including the side surfaces, is covered by coating B. The substrate platelets are thus completely enveloped by coating B. This improves the optical properties of the pigment and increases its mechanical and chemical resistance. The above also applies to layer A and preferably also to layer C, if present.

Although multiple coatings A, B and/or C may be present in each case, the coated substrate platelets preferably have only one coating A, B and, if present, C in each case.

The coating B is composed of at least one highly refractive metal oxide. Highly refractive materials have a refractive index of at least about 1.9, preferably at least about 2.0, and more preferably at least about 2.4. Preferably, the coating B comprises at least about 95 wt. %, more preferably at least about 99 wt. %, of high refractive index metal oxide(s).

The coating B has a thickness of at least about 50 nm. Preferably, the thickness of coating B is no more than about 400 nm, more preferably no more than about 300 nm.

Highly refractive metal oxides suitable for coating B are preferably selectively light-absorbing (i.e. colored) metal oxides, such as iron(III) oxide (α- and γ-Fe2O3, red), cobalt(II) oxide (blue), chromium(III) oxide (green), titanium(III) oxide (blue, usually present in admixture with titanium oxynitrides and titanium nitrides), and vanadium(V) oxide (orange), and mixtures thereof. Colorless high-index oxides such as titanium dioxide and/or zirconium oxide are also suitable.

Coating B may contain a selectively absorbing dye, preferably about 0.001 to about 5 wt. %, particularly preferably about 0.01 to about 1 wt. %, in each case based on the total amount of coating B. Suitable dyes are organic and inorganic dyes which can be stably incorporated into a metal oxide coating.

The coating A preferably has at least one low refractive index metal oxide and/or metal oxide hydrate. Preferably, coating A comprises at least about 95 wt. %, more preferably at least about 99 wt. %, of low refractive index metal oxide (hydrate). Low refractive index materials have a refractive index of about 1.8 or less, preferably about 1.6 or less.

Low refractive index metal oxides suitable for coating A include, for example, silicon (di)oxide, silicon oxide hydrate, aluminum oxide, aluminum oxide hydrate, boron oxide, germanium oxide, manganese oxide, magnesium oxide, and mixtures thereof, with silicon dioxide being preferred. The coating A preferably has a thickness of about 1 to about 100 nm, particularly preferably about 5 to about 50 nm, especially preferably about 5 to about 20 nm.

Preferably, the distance between the surface of the substrate platelets and the inner surface of coating B is at most about 100 nm, particularly preferably at most about 50 nm, especially preferably at most about 20 nm. By ensuring that the thickness of coating A, and thus the distance between the surface of the substrate platelets and coating B, is within the range specified above, it is possible to ensure that the pigments have a high hiding power.

If the pigment based on a lamellar substrate platelet has only one layer A, it is preferred that the pigment has a lamellar substrate platelet of aluminum and a layer A of silica. If the pigment based on a lamellar substrate platelet has a layer A and a layer B, it is preferred that the pigment has a lamellar substrate platelet of aluminum, a layer A of silica and a layer B of iron oxide.

According to a preferred embodiment, the pigments have a further coating C of a metal oxide (hydrate), which is different from the underlying coating B. Suitable metal oxides include silicon (di)oxide, silicon oxide hydrate, aluminum oxide, aluminum oxide hydrate, zinc oxide, tin oxide, titanium dioxide, zirconium oxide, iron (III) oxide, and chromium (III) oxide. Silicon dioxide is preferred.

The coating C preferably has a thickness of about 10 to about 500 nm, more preferably about 50 to about 300 nm. By providing coating C, for example based on TiO₂, better interference can be achieved while maintaining high hiding power.

Layers A and C serve in particular as corrosion protection as well as chemical and physical stabilization. Particularly preferred layers A and C are silica or alumina applied by the sol-gel process. This process comprises dispersing the uncoated lamellar substrate platelets or the lamellar substrate platelets already coated with layer A and/or layer B in a solution of a metal alkoxide such as tetraethyl orthosilicate or aluminum triisopropanolate (usually in a solution of organic solvent or a mixture of organic solvent and water with at least about 50% by weight of organic solvent such as a C₁ to C₄ alcohol), and adding a weak base or acid to hydrolyze the metal alkoxide, thereby forming a film of the metal oxide on the surface of the (coated) substrate platelets.

Layer B can be produced, for example, by hydrolytic decomposition of one or more organic metal compounds and/or by precipitation of one or more dissolved metal salts, as well as any subsequent post-treatment (for example, transfer of a formed hydroxide-containing layer to the oxide layers by annealing).

Although each of the coatings A, B and/or C may be composed of a mixture of two or more metal oxide(hydrate)s, each of the coatings is preferably composed of one metal oxide(hydrate).

The pigments based on coated lamellar or lenticular substrate platelets or the pigments based on coated VMP substrate platelets preferably have a thickness of about 70 to about 500 nm, particularly preferably about 100 to about 400 nm, especially preferably about 150 to about 320 nm, for example about 180 to about 290 nm. Due to the low thickness of the substrate platelets, the pigment exhibits particularly high hiding power. The low thickness of the coated substrate platelets is achieved in particular by keeping the thickness of the uncoated substrate platelets low, but also by adjusting the thicknesses of the coatings A and, if present, C to as small a value as possible. The thickness of coating B determines the color impression of the pigment.

The adhesion and abrasion resistance of pigments based on coated substrate platelets in keratinic material can be significantly increased by additionally modifying the outermost layer, layer A, B or C depending on the structure, with organic compounds such as silanes, phosphoric acid esters, titanates, borates or carboxylic acids. In this case, the organic compounds are bonded to the surface of the outermost, preferably metal oxide-containing, layer A, B, or C. The outermost layer denotes the layer that is spatially farthest from the lamellar substrate platelet. The organic compounds are preferably functional silane compounds that can bind to the metal oxide-containing layer A, B, or C. These can be either mono- or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3-methacryloxy-propyltriethoxysilane, 3-acryloxypropyltrimethoxysilane, 2-methacryloxyethyl-triethoxysilane, 2-acryloxyethyltriethoxysilane, 3-methacryloxypropyltris(methoxyethoxy)silane, 3-methacryloxypropyltris(butoxyethoxy)silane, 3-methacryloxy-propyltris(propoxy)silane, 3-methacryloxypropyltris(butoxy)silane, 3-acryloxy-propyltris(methoxyethoxy)silane, 3-acryloxypropyltris(butoxyethoxy)silane, 3-acryl-oxypropyltris(butoxy)silane, vinyltrimethoxysilane, vinyltriethoxysilane, vinylethyl dichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, in particular an alkylsilane or arylsilane, can be carried out. This has only one functional group, which can covalently bond to the surface pigment based on coated lamellar substrate platelets (i.e. to the outermost metal oxide-containing layer) or, if not completely covered, to the metal surface. The hydrocarbon residue of the silane points away from the pigment. Depending on the type and nature of the hydrocarbon residue of the silane, a varying degree of hydrophobicity of the pigment is achieved. Examples of such silanes include hexadecyltrimethoxysilane, propyltrimethoxysilane, etc. Particularly preferred are pigments based on silica-coated aluminum substrate platelets surface-modified with a monofunctional silane. Octyltrimethoxysilane, octyltriethoxysilane, hecadecyltrimethoxysilane and hecadecyltriethoxysilane are particularly preferred. Due to the changed surface properties/hydrophobization, an improvement can be achieved in terms of adhesion, abrasion resistance and alignment in the application.

Suitable pigments based on a lamellar substrate platelet include, for example, the pigments of the VISIONAIRE® series from Eckart®.

Pigments based on a lenticular substrate platelet are available, for example, under the name Alegrace® Gorgeous from the company Schlenk® Metallic Pigments GmbH.

Pigments based on a substrate platelet comprising a vacuum metallized pigment are available, for example, under the name Alegrace® Marvelous or Alegrace® Aurous from the company SchlenkSchlenk Metallic Pigments GmbH.

In a further embodiment, a process as contemplated herein is exemplified in that the composition (A) contains—based on the total weight of the composition (A)—one or more pigments in a total amount of from about 0.001 to about 20 wt. %, in particular from about 0.05 to about 5 wt. %.

In a further embodiment, a process as contemplated herein is exemplified in that the composition (B) contains—based on the total weight of the composition (B)—one or more pigments in a total amount of from about 0.001 to about 20 wt. %, in particular from about 0.05 to about 5 wt. %.

As coloring compounds, the compositions as contemplated herein may also contain one or more direct dyes. Direct dyes are dyes that are applied directly to the hair and do not require an oxidative process to develop the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments. Preferably, the direct dyes within the meaning of the present disclosure have a solubility in water (760 mmHg) at 25° C. of more than 1.0 g/L. In particular, the direct dyes within the meaning of the present disclosure have a solubility in water (760 mmHg) at 25° C. of more than 1.5 g/L.

Direct dyes can be divided into anionic, cationic and nonionic direct dyes.

In a further preferred embodiment, an agent as contemplated herein is exemplified in that it contains at least one anionic, cationic and/or nonionic direct dye as the coloring compound.

In a further preferred embodiment, a process as contemplated herein is exemplified in that the composition (B) and/or the composition (C) comprises at least one colorant compound selected from the group of anionic, nonionic, and/or cationic direct dyes.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulfonic acid group (—SO₃H). Depending on the pH value, the protonated forms (—COOH, —SO₃H) of the carboxylic acid or sulfonic acid groups are in equilibrium with their deprotonated forms (—COO⁻, —SO₃⁻ present). The proportion of protonated forms increases with decreasing pH. If direct dyes are used in the form of their salts, the carboxylic acid groups or sulfonic acid groups are present in deprotonated form and are neutralized with corresponding stoichiometric equivalents of cations to maintain electro neutrality. Acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. Preferably the acid dyes within the meaning of the present disclosure have a solubility in water (760 mmHg) at 25° C. of more than 1.0 g/L. The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential feature of acid dyes is their ability to form anionic charges, whereby the carboxylic acid or sulfonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA n° B001), Acid Yellow 3 (COLIPA n°: C 54, D&C Yellow N° 10, Quinoline Yellow, E104, Food Yellow 13), Acid Yellow 9 (CI 13015), Acid Yellow 17 (CI 18965), Acid Yellow 23 (COLIPA n° C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n° C.015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I.14720), Acid Red 18 (E124, Red 18; CI 16255), Acid Red 27 (E 123, CI 16185, C-Rot 46, Real red D, FD&C Red Nr. 2, Food Red 9, Naphthol red S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA n° C.53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet n° 2, C.I. 60730, COLIPA n° C.063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I.42100), Acid Green 22 (C.I.42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA n° B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. An agitator is added. Then add 100 ml of water. This mixture is heated to 25° C. on a magnetic stirrer while stirring. It is stirred for 60 minutes. The aqueous mixture is then visually assessed. If there are still undissolved radicals, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).
Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and readily soluble in water at 25° C.
Acid Orange 7 is the sodium salt of 4-[(2-hydroxy-1-naphthyl)azo]benzene sulfonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a very high water solubility of more than 20 wt. %.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl} {4-[(N-ethyl(3-sulfonatobenzyl)imino]-2,5-cyclohexadien-1-ylidene}methyl)-benzenesulfonate and has a solubility in water of more than 20 wt. % (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to reversibly or irreversibly change its color depending on irradiation with light, especially UV light. This can be done by changing both the intensity and/or the wavelength maximum.

### Multi-Component Packaging Unit (Kit-of-Parts)

To increase user convenience, all preparations required for the hair treatment process can be provided to the user in the form of a multi-component packaging unit (kit-of-parts).

A third object of the present disclosure is a multi-component packaging unit (kit-of-parts) for treating keratinous material, in particular human hair, which are separately assembled
a first packaging unit containing a cosmetic preparation (A) and
a second packaging unit containing a cosmetic preparation (B), where
the cosmetic preparation (A) comprises a mixture of organic C₁-C₆ alkoxy siloxanes, the preparation of which has been described above, and
the cosmetic preparation (B) is a water-containing cosmetic carrier which preferably contains at least one fatty constituent and/or at least one surfactant.

For the purposes of the present disclosure, “fatty components” means organic compounds with a solubility in water at room temperature (22° C.) and atmospheric pressure (760 mmHg) of less than 1 wt. %, preferably less than 0.1 wt. %. The definition of fat constituents explicitly covers only uncharged (i.e. non-ionic) compounds. Fat components have at least one saturated or unsaturated alkyl group with at least 12 C atoms. The molecular weight of the fat constituents is a maximum of about 5000 g/mol, preferably a maximum of about 2500 g/mol and particularly preferably a maximum of about 1000 g/mol. The fat components are neither polyoxyalkylated nor polyglycerylated compounds.

Very preferably, the fat components are selected from the group of C₁₂-C₃₀ fatty alcohols, C₁₂-C₃₀ fatty acid triglycerides, C₁₂-C₃₀ fatty acid monoglycerides, C₁₂-C₃₀ fatty acid diglycerides and/or hydrocarbons.

The C₁₂-C₃₀ fatty alcohols can be saturated, mono- or polyunsaturated, linear or branched fatty alcohols with about 12 to about 30 C atoms.

Examples of particularly preferred linear, saturated C₁₂-C₃₀ fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol). Preferred linear unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenoyl alcohol), gadoleyl alcohol ((9Z)-eicos-9-en-1-ol), arachidone alcohol ((5Z,8Z,11Z,14Z)-eicosa-5,8,11,14-tetraen-1-ol), erucyl alcohol ((13Z)-docos-13-en-1-ol), and/or brassidyl alcohol ((13E)-docosen-1-ol). The preferred representatives for branched fatty alcohols are 2-octyl-dodecanol, 2-hexyl-dodecanol and/or 2-butyl-dodecanol.

The term surfactants (T) refers to surface-active substances that can form adsorption layers on surfaces and interfaces or aggregate in bulk phases to form micelle colloids or lyotropic mesophases. A distinction is made between anionic surfactants of a hydrophobic radical and a negatively charged hydrophilic head group, amphoteric surfactants, which carry both a negative and a compensating positive charge, cationic surfactants, which have a positively charged hydrophilic group in addition to a hydrophobic radical, and nonionic surfactants, which have no charges but strong dipole moments and are strongly hydrated in aqueous solution.

In a very particularly preferred embodiment, a process as contemplated herein is exemplified in that the second preparation (B) comprises at least one nonionic surfactant.

Non-ionic surfactants contain, for example, a polyol group, a polyalkylene glycol ether group or a combination of polyol and polyglycol ether group as the hydrophilic group. Such links include

Addition products of about 2 to about 50 mol ethylene oxide and/or 0 to about 5 mol propylene oxide to linear and branched fatty alcohols with 6 to about 30 C atoms, the fatty alcohol polyglycol ethers or the fatty alcohol polypropylene glycol ethers or mixed fatty alcohol polyethers,
Addition products of about 2 to about 50 mol ethylene oxide and/or 0 to about 5 mol propylene oxide to linear and branched fatty acids with 6 to about 30 C atoms, the fatty acid polyglycol ethers or the fatty acid polypropylene glycol ethers or mixed fatty acid polyethers,
Addition products of about 2 to about 50 mol ethylene oxide and/or 0 to about 5 mol propylene oxide to linear and branched alkylphenols having about 8 to about 15 C atoms in the alkyl group, the alkylphenol polyglycol ethers or the alkylpolypropylene glycol ethers or mixed alkylphenol polyethers,
Addition products of about 2 to about 50 moles of ethylene oxide and/or 0 to about 5 moles of propylene oxide to linear and branched fatty alcohols having about 8 to about 30 carbon atoms, to fatty acids, end-capped with a methyl or C₂-C₆-alkyl radical about 8 to about 30 carbon atoms and on alkyl phenols with about 8 to about 15 carbon atoms in the alkyl group, such as the types available under the sales names Dehydol® LS and Dehydol® LT (Cognis), C₁₂-C₃₀ fatty acid mono- and diesters of addition products of about 1 to about 30 mol ethylene oxide to glycerol,
Addition products of about 5 to about 60 mol ethylene oxide to castor oil and hardened castor oil,
Polyol fatty acid esters, such as the commercial product Hydagen® HSP (Cognis) or Sovermol® grades (Cognis),
alkoxylated triglycerides,
alkoxylated fatty acid alkyl esters of the formula (Tnio-1)

R¹CO—(OCH₂CHR²)_{w}OR³ (Tnio-1)

in which R¹CO is a linear or branched, saturated and/or unsaturated acyl radical having 6 to about 22 carbon atoms, R² is hydrogen or methyl, R³ is linear or branched alkyl radicals having 1 to 4 carbon atoms and w is numbers from 1 to about 20,
amine oxides,
Hydroxy mixed ethers, as described for example in DE-OS 19738866,
Sorbitan fatty acid esters and addition products of ethylene oxide to sorbitan fatty acid esters such as polysorbates,
Sugar fatty acid esters and addition products of ethylene oxide to sugar fatty acid ester,
Addition products of ethylene oxide to fatty acid alkanolamides and fatty amines,
Sugar tensides of the alkyl and alkenyl oligoglycoside type according to formula (Tnio-2),

R⁴O-[G]ₚ (Tnio-2)

in which R⁴ is an alkyl or alkenyl radical containing 4 to about 22 carbon atoms, G is a sugar residue containing 5 or 6 carbon atoms and p is a number of 1 to about 10. They can be obtained by the relevant methods of preparative organic chemistry. The alkyl and alkenyl oligoglycosides can be derived from aldoses or ketoses with 5 or 6 carbon atoms, preferably glucose. The preferred alkyl and/or alkenyl oligoglycosides are thus alkyl and/or alkenyl oligoglucosides. The index number p in the general formula (Tnio-2) indicates the degree of oligomerization (DP), i.e. the distribution of mono- and oligoglycosides and stands for a number between about 1 and about 10. While p must always be an integer in the individual molecule and can assume the values p=1 to 6, the value p for a certain alkyl oligoglycoside is an analytically determined arithmetical quantity, which usually represents a fractional number. Preferably alkyl and/or alkenyl oligoglycosides with an average degree of oligomerization p of about 1.1 to about 3.0 are used. From an application technology point of view, those alkyl and/or alkenyl oligoglycosides are preferred whose degree of oligomerization is less than about 1.7 and in particular lies between about 1.2 and about 1.4. The alkyl or alkenyl radical R⁴ can be derived from primary alcohols containing 4 to about 11, preferably about 8 to about 10 carbon atoms. Typical examples are butanol, caproic alcohol, caprylic alcohol, caprin alcohol and undecrylic alcohol as well as their technical mixtures, such as those obtained in the hydrogenation of technical fatty acid methyl esters or in the course of the hydrogenation of aldehydes from Roelen's oxo synthesis. Preferred are alkyl oligoglucosides with a chain length of C₈-C₁₀ (DP=1 to 3), which are obtained as a preliminary step in the distillative separation of technical C₈-C₁₈ coconut-fatty alcohol and may be contaminated with less than about 6 wt. % of C₁₂ alcohol, and alkyl oligoglucosides based on technical C_{9/11} oxoalcohols (DP=about 1 to about 3). The alkyl or alkenyl radical R¹⁵ can also be derived from primary alcohols having about 12 to about 22, preferably about 12 to about 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol and their technical mixtures, which can be obtained as described above. Preferred are alkyl oligoglucosides based on hardened C_{12/14} coconut alcohol with a DP of about 1 to about 3.
Sugar surfactants of the fatty acid N-alkyl polyhydroxyalkylamide type, a nonionic surfactant of formula (Tnio-3)

R⁵CO—NR⁶—[Z] (Tnio-3)

in which R⁵CO is an aliphatic acyl radical containing 6 to about 22 carbon atoms, R⁶ is hydrogen, an alkyl or hydroxyalkyl radical containing 1 to 4 carbon atoms and [Z] is a linear or branched polyhydroxyalkyl radical containing 3 to about 12 carbon atoms and about 3 to about 10 hydroxyl groups. The fatty acid N-alkyl polyhydroxyalkylamides are known substances that can usually be obtained by reductive amination of a reducing sugar with ammonia, an alkylamine or an alkanolamine and subsequent acylation with a fatty acid, a fatty acid alkyl ester or a fatty acid chloride. The fatty acid N-alkyl polyhydroxyalkylamides are preferably derived from reducing sugars with 5 or 6 carbon atoms, especially from glucose. The preferred fatty acid N-alkyl polyhydroxyalkylamides are therefore fatty acid N-alkylglucamides as represented by the formula (Tnio-4):

R⁷CO—(NR⁸)—CH₂—[CH(OH)]₄—CH₂OH (Tnio-4)

Preferably, glucamides of the formula (Tnio-4) are used as fatty acid-N-alkyl polyhydroxyalkylamides, in which R⁸ represents hydrogen or an alkyl group and RICO represents the acyl radical of caproic acid, caprylic acid, capric acid, Lauric acid, myristic acid, palmitic acid, palmoleic acid, stearic acid, isostearic acid, oleic acid, elaidic acid, petroselinic acid, linoleic acid, linolenic acid, arachidic acid, gadoleic acid, behenic acid or erucic acid or their technical mixtures. Particularly preferred are fatty acid N-alkyl glucamides of the formula (Tnio-4), which are obtained by reductive amination of glucose with methylamine and subsequent acylation with lauric acid or about C12/14 coconut fatty acid or a corresponding derivative. Furthermore, polyhydroxyalkylamides can also be derived from maltose and palatinose.

The fatty constituent(s) and the surfactant(s) may be present in the preparation (B) in amounts ranging from about 0.1 to about 20% by weight, preferably from about 1.0 to about 10.0% by weight, based on the total weight of the preparation (B).

With regard to the further preferred embodiments of the multicomponent packaging unit as contemplated herein’, what has been said about the processes applies mutatis mutandis.

### Examples

1. Preparation of Mixtures of Organic C₁-C₆ Alkoxy Siloxanes

### 1.1. Siloxane Mixture 1—Acid Catalysis

In a 500 ml round bottom flask, 25 g ethanol (abs.) and 49.7 g methyltriethoxysilane were mixed together with stirring. This mixture was heated to 50° C. with further stirring.
Then 6.8 g of a 1% solution of sulfuric acid in water was added over a period of about 5 minutes. The temperature of the reaction mixture increased to 62° C. and decreased to 55° C. after the addition was completed. Stirring was continued for another 20 minutes. Then 18.6 g of (3-aminopropyl)triethoxysilane was added over a period of about 5 minutes. After completion of the addition, the mixture was stirred at 50° C. for an additional 45 minutes and subsequently poured into an airtight glass jar.

### 1.2. Siloxane Mixture 2—Acid Catalysis

In a 500 ml round bottom flask, 21.4 g ethanol (abs.) and 52.9 g ethyltriethoxysilane were mixed together with stirring. This mixture was heated to 50° C. with further stirring.

Then 9.6 g of a 1% solution of sulfuric acid in water was added over a period of about 5 minutes. The temperature of the reaction mixture increased to 61° C. and decreased to 55° C. after the addition was completed. Stirring was continued for another 25 minutes. Then 16.0 g of (3-aminopropyl)triethoxysilane was added over a period of about 5 minutes. After completion of the addition, the mixture was stirred at 50° C. for an additional 45 minutes and subsequently poured into an airtight glass jar.

### 1.3. Siloxane Mixture 3—Acid Catalysis

In a 500 ml round bottom flask, 22.2 g ethanol (abs.) and 55.0 g ethyltriethoxysilane were mixed together with stirring. This mixture was heated to 50° C. with further stirring.

Then 6.1 g of a 1% solution of sulfuric acid in water was added over a period of about 5 minutes. The temperature of the reaction mixture increased to 61° C. and decreased to 55° C. after the addition was completed. Stirring was continued for another 20 minutes. Then 16.7 g of (3-aminopropyl)triethoxysilane was added over a period of about 5 minutes. After completion of the addition, the mixture was stirred at 50° C. for an additional 45 minutes and subsequently poured into an airtight glass jar.

### 1.4. Siloxane Mixture 4—Acid Catalysis

In a 500 ml round bottom flask, 22.6 g ethanol (abs.) and 54.2 g ethyltriethoxysilane were mixed together with stirring. This mixture was heated to 50° C. with further stirring.

Then 6.2 g of a 1% solution of sulfuric acid in water was added over a period of about 5 minutes. The temperature of the reaction mixture increased to 61° C. and decreased to 55° C. after the addition was completed. Stirring was continued for another 20 minutes. Then 16.9 g of (3-aminopropyl)triethoxysilane was added over a period of about 5 minutes. After completion of the addition, the mixture was stirred at 50° C. for an additional 45 minutes and subsequently poured into an airtight glass jar.

### 1.5. Siloxane Mixture 5—Base Catalysis

In a 500 ml round bottom flask, 23.4 g ethanol (abs.) and 52.6 g methyltriethoxysilane and 17.5 g 3-aminopropyl)triethoxysilane were mixed together under stirring. This mixture was heated to 50° C. with further stirring. Then 6.4 g of a 1% solution of sodium hydroxide in water was added over a period of about 5 minutes. The temperature of the reaction mixture increased to 59° C. and decreased to 55° C. after the addition was completed. The mixture was stirred at 50° C. for a further 45 minutes and subsequently poured into an airtight glass vessel.

### 3. Coloring

The following colorants were provided:

Preparation (A), mixture of organic C₁-C₆ alkoxy siloxanes

| | |
|---|---|
| Siloxane mixture 1 | 10 g |
| Siloxane mixture 2 | 10 g |
| Siloxane mixture 3 | 10 g |
| Siloxane mixture 4 | 10 g |
| Siloxane mixture 5 | 10 g |

### Preparation (B)

| | | |
|---|---|---|
| Colorona ® Bordeaux, Merck ®, MICA, CI 77491 | 5.5 | g |
| (IRON OXIDES) | | |
| Hydroxyethyl cellulose (Natrosol ® 250 HR) | 1.0 | g |
| Cetearyl alcohol | 1.5 | g |
| Eumulgin ® B3 (Ceteareth-30) | 1.5 | g |
| Water | Ad 100 | g |

The ready-to-use stain was prepared by shaking 10 g of preparation (A) and 100 g of preparation (B), respectively (shaking for 3 minutes). Then one strand of hair (Kerling Euronaturhaar white) was dipped into the ready-to-use dye and left in it for 1 minute. After that, superfluous agent was striped from each strand of hair. Subsequently, each strand of hair was washed with water and dried. Subsequently, the strands were visually evaluated under a daylight lamp. The following results were obtained:

| | |
|---|---|
| Preparation (A), Siloxane mixture 1 + | burgundy red, high |
| Preparation (B) | color intensity |
| Preparation (A), Siloxane mixture 2 + | burgundy red, high |
| Preparation (B) | color intensity |
| Preparation (A), Siloxane mixture 3 + | burgundy red, high |
| Preparation (B) | color intensity |
| Preparation (A), Siloxane mixture 4 + | burgundy red, high |
| Preparation (B) | color intensity |
| Preparation (A), Siloxane mixture 5 + | burgundy red, high |
| Preparation (B) | color intensity |

1. A method for the treatment of keratinous material, the method comprising the steps of:
applying a cosmetic agent to the keratinous material, wherein the cosmetic agent comprises an organic C₁-C₆ alkoxy siloxane which is obtained by mixing an organic C₁-C₆ alkoxy silane with a solvent other than water and selectively hydrolyzing and precondensing the organic C₁-C₆ alkoxy silane to form the organic C₁-C₆ alkoxy siloxane by adding water and a catalyst; and
rinsing the cosmetic agent off of the keratinous material after a contact time.

2. The method according to claim 1, wherein the cosmetic agent comprises the organic C₁-C₆ alkoxy siloxane which is obtained by mixing one or more of the organic C₁-C₆ alkoxy silanes of formula (I) and/or (II) and/or (IV) with the solvent
R₁R₂N-L-Si(OR₃)ₐ(R₄)_{b} (I)
where
R₁, R₂ independently represent a hydrogen atom or a C₁-C₆ alkyl group,
L is a linear or branched divalent C₁-C₂₀ alkylene group,
R₃, R₄ independently of one another represent a C₁-C₆ alkyl group,
a stands for an integer from 1 to 3, and
b stands for the integer 3-a, and
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
where
R₅, R_{5′}, R₆, and R_{6′}, independently represent a C₁-C₆ alkyl group,
A, A′, A″, and A′″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III),
-(A″″)-Si(R₆″)_{d}″(OR₅″)_{c}″ (III),
R_{5″} and R_{6″} independently represent a C₁-C₆ alkyl group,
c stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h radicals are different from 0,
(R₉)ₘSi(OR₁₀)ₖ (IV),
where
R₉ is a C₁-C₁₂ alkyl group or a C₂-C₁₂ alkenyl group,
R₁₀ represents a C₁-C₆ alkyl group,
k is an integer from 1 to 4, and
m stands for the number 4-k.

3. The method according to claim 1, wherein the cosmetic agent comprises the organic C₁-C₆ alkoxy siloxane obtained by mixing with the solvent one or more of the organic C₁-C₆ alkoxy silanes selected from the group of:
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane,
Dodecyltriethoxysilane,
Vinyl trimethoxysilane
Vinyl triethoxysilane
Tetramethoxysilane
Tetraethoxysilane
(3-Aminopropyl)triethoxysilane
(3-Aminopropyl)trimethoxysilane
(2-Aminoethyl)triethoxysilane
(2-Aminoethyl)trimethoxysilane
(3-Dimethylaminopropyl)triethoxysilane
(3-Dimethylaminopropyl)trimethoxysilane
(2-dimethylaminoethyl)triethoxysilane,
(2-Dimethylaminoethyl)trimethoxysilane, and
combinations thereof.

4. The method according to claim 2, wherein the cosmetic agent comprises the organic C₁-C₆ alkoxy siloxanes obtained by mixing one or more organic C₁-C₆ alkoxy silanes of formula (I) and one or more organic C₁-C₆ alkoxysilanes of formula (IV) in a weight ratio of (I)/(IV) of about 1:1 to about 1:10 to each other.

5. The method according to claim 1, wherein the solvent other than water is selected from the group of mono- or polyhydric C₁-C₁₂ alcohols.

6. The method according to claim 1, wherein the cosmetic agent comprises the organic C₁-C₆ alkoxy siloxanes obtained by combining the organic C₁-C₆ alkoxy silane and the solvent in a weight ratio of from about 5:1 to about 1:5.

7. The method according to claim 1, further comprising heating the organic C₁-C₆ alkoxy silane and the solvent to a temperature of from about 30 to about 80° C. before the water and the catalyst are added.

8. The method according to claim 1, wherein the organic C₁-C₆ alkoxy silane is selectively hydrolyzed by adding from about 0.10 to about 0.80 molar equivalents of the water (S-W), wherein the molar equivalents of the water (S-W) are calculated according to the formula
$$S - W = \frac{mol ( Water )}{mol ( Silane ) \times n ( Alkoxy )}$$
wherein
S-W=molar equivalents of the water
mol(water)=molar quantity of the water used
mol(silanes)=total molar amount of the organic C₁-C₆ alkoxy silanes used in the reaction
n(Alkoxy)=Number of C₁-C₆ alkoxy groups per the organic C₁-C₆ alkoxy silane.

9. The method of claim 1, wherein the catalyst is selected from the group of inorganic and organic acids.

10. The method according to claim 1, wherein the catalyst is selected from the group of inorganic and organic bases.

11. The method according to claim 2, wherein the cosmetic agent comprises the organic C₁-C₆ alkoxy siloxane obtained by
(1) the organic C₁-C₆ alkoxy silane of the formula (IV) are mixed with the solvent other than water and selectively hydrolyzed and precondensed by adding the water and the catalyst,
(2) a mixture obtained in step (1) is stirred for a period of from about 5 minutes to about 3 hours at a temperature of from about 30 to about 80° C., and then
(3) a mixture obtained in step (2) is mixed with the organic C₁-C₆ alkoxy silane of formula (I).

12. The method according to claim 11, wherein:
(4) a mixture obtained in step (3) is stirred at a temperature of from about 30 to about 80° C. for a period of from about 20 minutes to about 24 hours.

13. The method according to claim 2, wherein the cosmetic agent comprises the organic C₁-C₆ alkoxy siloxane obtained by
(1) mixing the organic C₁-C₆ alkoxy silane of the formula (I) with the organic C₁-C₆ alkoxy silane of the formula (IV) and the solvent other than water and are selectively hydrolyzed and precondensed by adding the water and the catalyst.

14. The method according to claim 13, wherein:
(2) a mixture obtained in step (1) is stirred at a temperature of from about 30 to about 80° C. for a period of from about 20 hours to about 3 days.

15. A method for treating keratinous material, the method comprising the following steps in the order indicated:
(i) providing a mixture of organic C₁-C₆ alkoxy siloxanes, wherein the mixture of the organic C₁-C₆ alkoxy siloxanes is obtained by mixing an organic C₁-C₆ alkoxy silane with a solvent other than water and selectively hydrolyzing and precondensing the organic C₁-C₆ alkoxy silane to form the organic C₁-C₆ alkoxy siloxane by adding water and a catalyst,
(ii) blending the mixture of the C₁-C₆ organic alkoxy siloxanes with a water-containing cosmetic carrier to obtain a ready-to-use cosmetic agent,
(iii) applying the ready-to-use cosmetic agent prepared in step (ii) to the keratinous material,
(iv) exposing the ready-to-use cosmetic agent applied in step (iii) to the keratinous material,
(v) rinsing the ready-to-use cosmetic agent from the keratinous material,
(vi) optionally applying a post-treatment agent to the keratinous material,
(vii) optionally exposing the keratinous material to the post-treatment agent; and
(viii) optionally rinsing the post-treatment agent off the keratinous material.

16. The method according to claim 15, wherein the method is a method for coloring human hair and the ready-to-use cosmetic agent applied in step (iii) additionally comprises a pigment and/or a direct dye.

17. The method according to claim 15, wherein the method is a method for dyeing human hair and the post-treatment agent applied in step (vi) comprises a pigment and/or a direct dye.

18. A multicomponent packaging unit (kit-of-parts) for the treatment of keratinous material, comprising:
a first packaging unit containing a cosmetic preparation (A) and
a second packaging unit containing a cosmetic preparation (B),
where
the cosmetic preparation (A) comprises an organic C₁-C₆ alkoxy siloxane which is obtained by mixing an organic C₁-C₆ alkoxy silane with a solvent other than water and selectively hydrolyzing and precondensing the organic C₁-C₆ alkoxy silane to form the organic C₁-C₆ alkoxy siloxane by adding water and a catalyst, and
the cosmetic preparation (B) comprises a water-containing cosmetic carrier.

19. The method of claim 1, wherein the catalyst is selected from the group of sulfuric acid, hydrochloric acid, phosphoric acid, maleic acid, citric acid, tartaric acid, malic acid, lactic acid, acetic acid, methanesulfonic acid, benzoic acid, malonic acid, oxalic acid, 1-hydroxyethane-1,1-diphosphonic acid, sodium hydroxide, potassium hydroxide, magnesium hydroxide, calcium hydroxide, and combinations thereof.

20. The method of claim 1, wherein the solvent is selected from the group of methanol, ethanol, and isopropanol.
